(19) United States
(12) Patent Application Publication
(10) Pub. No.: US 2009/0318369 A1
(43) Pub. Date: Dec. 24, 2009

(54) BIOMARKERS FOR ALZHEIMER'S DISEASE AND METHODS USING THE SAME

(76) Inventors: Lisa A. Paige, Hillsborough, NC (US); Matthew W. Mitchell, Durham, NC (US); Anne Evans, Durham, NC (US); Don Harvan, Durham, NC (US)

Correspondence Address: BRINKS, HOFER, GILSON & LIONE, P.O. BOX 1340, MORRISVILLE, NC, 27560, US

(21) Appl. No.: 12/192,469

(22) Filed: Aug. 15, 2008

Related U.S. Application Data
(60) Provisional application No. 60/956,486, filed on Aug. 17, 2007.

(51) Int. Cl. A61K 31/70; G01N 33/50; G01N 33/92; G01N 33/53; C12Q 1/02; G01N 33/68; A61K 31/195; A61K 31/56; A61K 31/19; A61K 31/415; A61K 31/52; A61K 31/66; A61P 25/28

(52) U.S. Cl. 514/23; 436/111; 436/71; 436/129; 436/96; 436/98; 436/93; 436/103; 436/94; 435/7.92; 435/29; 436/86; 514/563; 514/182; 514/557; 514/396; 514/263.1; 514/75

(57) ABSTRACT

The present invention provides various biomarkers of Alzheimer's Disease (AD). The present invention also provides various methods of using the biomarkers, including methods for diagnosis of AD, methods of determining predisposition to AD, methods of monitoring progression/regression of AD, methods of assessing efficacy of compositions for treating AD, methods of screening compositions for activity in modulating biomarkers of AD, methods of treating AD, as well as other methods based on biomarkers of AD.

### CROSS-REFERENCE TO RELATED APPLICATIONS

This application claims the benefit of U.S. Provisional Application No. 60/956,486, filed Aug. 17, 2007, the entirety of which is hereby incorporated by reference herein.

### FIELD

The invention generally relates to biomarkers for Alzheimer's Disease and methods based on the same biomarkers.

### BACKGROUND

Alzheimer's disease (AD) is a progressive brain disorder that over time destroys the memory, as well as the ability to learn, reason, make judgments, communicate and carry out activities of daily living. As the disease progresses, patients may experience behavior and personality changes such as suspiciousness, agitation, anxiety, delusions and/or hallucinations. The disease duration may vary from 3 to over 20 years. Eventually the patient requires complete care. An estimated 4.5 million people in the United States have AD, and the number continues to grow. The age of onset of AD is typically over 65 years of age, with the likelihood of developing AD doubling every five years after age 65. The risk of AD at age 85 is nearly 50 percent. In addition, AD can have an early on-set, striking both men and women younger than 65.

There is currently no single test for the diagnosis of AD. Presently, diagnosis typically involves a thorough medical history and physical examination including tests to assess memory and overall function of the mind and nervous system. A major goal of the diagnostic workup is to distinguish AD from other conditions with similar symptoms, such as depression, medication side effects, certain thyroid conditions, excess alcohol use, and nutritional imbalances, which are potentially treatable disorders that sometimes impair memory or other mental functions. Definitive diagnosis of AD using known methods can only be made post-mortem by analyzing the brain for presence of amyloid plaques and neuronal tangles.

### SUMMARY

The present invention provides methods for diagnosing whether a subject has Alzheimer's Disease (AD). Such methods comprise the steps of: analyzing a biological sample from a subject to determine the level(s) of one or more biomarkers for Alzheimer's Disease in the sample, where the one or more biomarkers are selected from Tables 1, 2, and/or 3; and comparing the level(s) of the one or more biomarkers in the sample to Alzheimer's Disease-positive and/or Alzheimer's Disease-negative reference levels of the one or more biomarkers in order to diagnose whether the subject has Alzheimer's Disease.

The present invention also provides methods of determining whether a subject is predisposed to developing Alzheimer's Disease (AD). Such methods comprise the steps of: analyzing a biological sample from a subject to determine the level(s) of one or more biomarkers for Alzheimer's Disease in the sample, where the one or more biomarkers are selected from Tables 1, 2, and/or 3; and comparing the level(s) of the one or more biomarkers in the sample to Alzheimer's Disease-positive and/or Alzheimer's Disease-negative reference levels of the one or more biomarkers in order to determine whether the subject is predisposed to developing Alzheimer's Disease.

The present invention further provides methods of monitoring progression/regression of Alzheimer's Disease (AD) in a subject. Such methods comprise: analyzing a first biological sample from a subject to determine the level(s) of one or more biomarkers for Alzheimer's Disease in the sample, where the one or more biomarkers are selected from Tables 1, 2, and/or 3 and the first sample is obtained from the subject at a first time point; analyzing a second biological sample from a subject to determine the level(s) of the one or more biomarkers, where the second sample is obtained from the subject at a second time point; and comparing the level(s) of one or more biomarkers in the first sample to the level(s) of the one or more biomarkers in the second sample in order to monitor the progression/regression of Alzheimer's Disease in the subject. Such methods may further comprise comparing the level(s) of one or more biomarkers in the first sample, the level(s) of one or more biomarkers in the second sample, and/or the results of the comparison of the level(s) of the one or more biomarkers in the first and second samples to Alzheimer's Disease-positive and/or Alzheimer's Disease-negative reference levels of the one or more biomarkers.

The present invention also provides methods of assessing the efficacy of a composition for treating Alzheimer's Disease (AD). Such methods comprise the steps of: analyzing, from a subject having Alzheimer's Disease and currently or previously being treated with a composition, a biological sample to determine the level(s) of one or more biomarkers for Alzheimer's Disease selected from Tables 1, 2, and/or 3; and comparing the level(s) of the one or more biomarkers in the sample to (a) levels of the one or more biomarkers in a previously-taken biological sample from the subject, where the previously-taken biological sample was obtained from the subject before being treated with the composition, (b) Alzheimer's Disease-positive reference levels of the one or more biomarkers, and/or (c) Alzheimer's Disease-negative reference levels of the one or more biomarkers.

The present invention further provides methods for assessing the efficacy of a composition in treating Alzheimer's Disease (AD). Such methods comprise: analyzing a first biological sample from a subject to determine the level(s) of one or more biomarkers for Alzheimer's Disease selected from Tables 1, 2, and/or 3, the first sample obtained from the subject at a first time point; administering the composition to the subject; analyzing a second biological sample from the subject to determine the level(s) of the one or more biomarkers, the second sample obtained from the subject at a second time point after administration of the composition; comparing the level(s) of one or more biomarkers in the first sample to the level(s) of the one or more biomarkers in the second sample in order to assess the efficacy of the composition for treating Alzheimer's Disease.

Methods of assessing the relative efficacy of two or more compositions for treating Alzheimer's Disease (AD) are also provided. Such methods comprise the steps of: analyzing, from a first subject having Alzheimer's Disease and currently or previously being treated with a first composition, a first biological sample to determine the level(s) of one or more biomarkers selected from Tables 1, 2, and/or 3; analyzing, from a second subject having Alzheimer's Disease and currently or previously being treated with a second composition, a second biological sample to determine the level(s) of the one or more biomarkers; and comparing the level(s) of one or more biomarkers in the first sample to the level(s) of the one or more biomarkers in the second sample in order to assess the relative efficacy of the first and second compositions for treating Alzheimer's Disease.

The present invention further provides methods for screening a composition for activity in modulating one or more biomarkers of Alzheimer's Disease, comprising: contacting one or more cells with a composition; analyzing at least a portion of the one or more cells or a biological sample associated with the cells to determine the level(s) of one or more biomarkers of Alzheimer's Disease selected from Tables 1, 2, and/or 3; and comparing the level(s) of the one or more biomarkers with predetermined standard levels for the biomarkers to determine whether the composition modulated the level(s) of the one or more biomarkers.

The present invention also provides methods for identifying a potential drug target for Alzheimer's Disease (AD). Such methods comprise the steps of: identifying one or more biochemical pathways associated with one or more biomarkers for Alzheimer's Disease selected from Tables 1, 2, and/or 3; and identifying a protein affecting at least one of the one or more identified biochemical pathways, the protein being a potential drug target for Alzheimer's Disease.

Also provided are methods for treating a subject having Alzheimer's Disease (AD) comprising administering to the subject an effective amount of one or more biomarkers selected from Tables 1, 2, and/or 3 that are decreased in Alzheimer's Disease.

### DETAILED DESCRIPTION

The present invention relates to biomarkers of Alzheimer's Disease, methods for diagnosis of Alzheimer's Disease, methods of determining predisposition to Alzheimer's Disease, methods of monitoring progression/regression of Alzheimer's Disease, methods of assessing efficacy of compositions for treating Alzheimer's Disease, methods of screening compositions for activity in modulating biomarkers of Alzheimer's Disease, methods of treating Alzheimer's Disease, as well as other methods based on biomarkers of Alzheimer's Disease. Prior to describing this invention in further detail, however, the following terms will first be defined.

### Definitions:

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological tissue or fluid such as, for example, blood, blood plasma, urine, or cerebral spinal fluid (CSF).

“Subject” means any animal, but is preferably a mammal, such as, for example, a human, monkey, non-human primate, rat, mouse, cow, dog, cat, pig, horse, or rabbit.

A “reference level” of a biomarker means a level of the biomarker that is indicative of a particular disease state, phenotype, or lack thereof, as well as combinations of disease states, phenotypes, or lack thereof. A “positive” reference level of a biomarker means a level that is indicative of a particular disease state or phenotype. A “negative” reference level of a biomarker means a level that is indicative of a lack of a particular disease state or phenotype. For example, an “Alzheimer's Disease-positive reference level” of a biomarker means a level of a biomarker that is indicative of a positive diagnosis of Alzheimer's Disease in a subject, and an “Alzheimer's Disease-negative reference level” of a biomarker means a level of a biomarker that is indicative of a negative diagnosis of Alzheimer's Disease in a subject. As another example, an “Alzheimer's-Disease-progression-positive reference level” of a biomarker means a level of a biomarker that is indicative of progression of Alzheimer's Disease in a subject, and an “Alzheimer's-Disease-regression-positive reference level” of a biomarker means a level of a biomarker that is indicative of regression of the Alzheimer's Disease. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched so that comparisons may be made between biomarker levels in samples from subjects of a certain age and reference levels for a particular disease state, phenotype, or lack thereof in a certain age group). Such reference levels may also be tailored to specific techniques that are used to measure levels of biomarkers in biological samples (e.g., LC-MS, GC-MS, etc.), where the levels of biomarkers may differ based on the specific technique that is used.

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolome” means all of the small molecules present in a given organism.

A “neurodegenerative disease” includes, but is not limited to, Alzheimer's Disease, amyotrophic lateral sclerosis, multiple sclerosis, Huntington's Disease, and Parkinson's Disease.

### I. Biomarkers

The Alzheimer's Disease biomarkers described herein were discovered using metabolomic profiling techniques. Such metabolomic profiling techniques are described in more detail in the Examples set forth below as well as in U.S. Pat. No. 7,005,255 and U.S. patent application Ser. Nos. 11/357,732, 10/695,265 (Publication No. 2005/0014132), Ser. No. 11/301,077 (Publication No. 2006/0134676), Ser. No. 11/301,078 (Publication No. 2006/0134677), Ser. No. 11/301,079 (Publication No. 2006/0134678), and Ser. No. 11/405,033, the entire contents of which are hereby incorporated herein by reference.

Generally, metabolic profiles were determined for biological samples from human subjects diagnosed with Alzheimer's Disease as well as from one or more other groups of human subjects (e.g., healthy control subjects not diagnosed with Alzheimer's Disease). The metabolic profile for Alzheimer's Disease was compared to the metabolic profile for biological samples from the one or more other groups of subjects. Those molecules differentially present, including those molecules differentially present at a level that is statistically significant, in the metabolic profile of Alzheimer's Disease samples as compared to another group (e.g., healthy control subjects not diagnosed with Alzheimer's Disease) were identified as biomarkers to distinguish those groups.

The biomarkers are discussed in more detail herein. The biomarkers that were discovered correspond with the following group(s):
Biomarkers for distinguishing Alzheimer's Disease vs. control subjects not diagnosed with Alzheimer's Disease (see Tables 1, 2, and 3).

Non-biomarker compounds associated with the compared groups were also identified. The non-biomarker compounds that were discovered correspond with the following group(s):
Non-biomarker compounds present at the same levels between Alzheimer's Disease and control subjects not diagnosed with Alzheimer's Disease (see Table 1).

Any number of biomarkers may be used in the methods disclosed herein. That is, the disclosed methods may include the determination of the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, fifteen or more biomarkers, etc., including a combination of all of the biomarkers in each or all of Tables 1, 2, 3, or any fraction thereof. In another aspect, the number of biomarkers for use in the disclosed methods include the levels of about thirty or less biomarkers, twenty-five or less, twenty or less, fifteen or less, ten or less, nine or less, eight or less, seven or less, six or less, five or less biomarkers. In another aspect, the number of biomarkers for use in the disclosed methods include the levels of one, two, three, four, five, six, seven, eight, nine, ten, eleven, twelve, thirteen, fourteen, fifteen, twenty, twenty-five, or thirty biomarkers.

Although the identities of some of the biomarkers and non-biomarker compounds are not known at this time, such identities are not necessary for the identification of the biomarkers or non-biomarker compounds in biological samples from subjects, as the “unnamed” compounds have been sufficiently characterized by analytical techniques to allow such identification. The analytical characterization of all such “unnamed” compounds is listed in the Examples. Such “unnamed” biomarkers and non-biomarker compounds are designated herein using the nomenclature “Metabolite” followed by a specific metabolite number.

In addition, the methods disclosed herein using the biomarkers listed in the tables may be used in combination with clinical diagnostic measures of Alzheimer's Disease and/or other neurodegenerative diseases. Combinations with clinical diagnostics may facilitate the disclosed methods, or confirm results of the disclosed methods (for example, facilitating or confirming diagnosis, monitoring progression or regression, and/or determining predisposition to Alzheimer's Disease).

Finally, where the potential identity of a compound is proposed for an “unnamed” metabolite and such identity has not been confirmed, the nomenclature of “possible” or “probable” (along with the potential compound identity) follows the “Metabolite” number. Such proposed identity should not be considered as limiting the analytical characterization of the otherwise “unnamed” compounds.

### II. Diagnosis of Alzheimer's Disease

The identification of biomarkers for Alzheimer's Disease allows for the diagnosis of (or for aiding in the diagnosis of) Alzheimer's Disease in subjects presenting one or more symptoms of Alzheimer's Disease. A method of diagnosing (or aiding in diagnosing) whether a subject has Alzheimer's Disease comprises (1) analyzing a biological sample from a subject to determine the level(s) of one or more biomarkers of Alzheimer's Disease in the sample and (2) comparing the level(s) of the one or more biomarkers in the sample to Alzheimer's Disease-positive and/or Alzheimer's Disease-negative reference levels of the one or more biomarkers in order to diagnose (or aid in the diagnosis of) whether the subject has Alzheimer's Disease. The one or more biomarkers that are used are selected from Tables 1, 2, and/or 3 and combinations thereof. When such a method is used to aid in the diagnosis of Alzheimer's Disease, the results of the method may be used along with other methods (or the results thereof) useful in the clinical determination of whether a subject has Alzheimer's Disease.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers of Tables 1, 2, and/or 3, may be determined in the methods of diagnosing and methods of aiding in diagnosing whether a subject has Alzheimer's Disease. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 2, and/or 3 or any fraction thereof, may be determined and used in such methods. Determining levels of combinations of the biomarkers may allow greater sensitivity and specificity in diagnosing Alzheimer's Disease and aiding in the diagnosis of Alzheimer's Disease, and may allow better differentiation of Alzheimer's Disease from other neurodegenerative diseases that may have similar or overlapping biomarkers to Alzheimer's Disease (as compared to a subject not having a neurodegenerative disease). For example, ratios of the levels of certain biomarkers (and non-biomarker compounds) in biological samples may allow greater sensitivity and specificity in diagnosing Alzheimer's Disease and aiding in the diagnosis of Alzheimer's Disease, and may allow better differentiation of Alzheimer's Disease from other neurodegenerative diseases that may have similar or overlapping biomarkers to Alzheimer's Disease (as compared to a subject not having a neurodegenerative disease).

One or more biomarkers that are specific for diagnosing Alzheimer's Disease (or aiding in diagnosing Alzheimer's Disease) in a subject within a certain group (e.g., females over 70 years of age) may also be used. For example, when the subject is a female over 70 years of age, one or more biomarkers listed in Table 2 may be used to diagnose (or aid in diagnosing) whether the subject has Alzheimer's Disease. When the subject is a male under 70 years of age, one or more biomarkers listed in Table 3 may be used to diagnose (or aid in diagnosing) whether the subject has Alzheimer's Disease.

After the level(s) of the one or more biomarkers in the sample are determined, the level(s) are compared to Alzheimer's Disease-positive and/or Alzheimer's Disease-negative reference levels to aid in diagnosing or to diagnose whether the subject has Alzheimer's Disease. Levels of the one or more biomarkers in a sample corresponding to the Alzheimer's Disease-positive reference levels (e.g., levels that are the same as the reference levels, substantially the same as the reference levels, above and/or below the minimum and/or maximum of the reference levels, and/or within the range of the reference levels) are indicative of a diagnosis of Alzheimer's Disease in the subject. Levels of the one or more biomarkers in a sample corresponding to the Alzheimer's Disease-negative reference levels (e.g., levels that are the same as the reference levels, substantially the same as the reference levels, above and/or below the minimum and/or maximum of the reference levels, and/or within the range of the reference levels) are indicative of a diagnosis of no Alzheimer's Disease in the subject. In addition, levels of the one or more biomarkers that are differentially present (especially at a level that is statistically significant) in the sample as compared to Alzheimer's Disease-negative reference levels are indicative of a diagnosis of Alzheimer's Disease in the subject. Levels of the one or more biomarkers that are differentially present (especially at a level that is statistically significant) in the sample as compared to Alzheimer's Disease-positive reference levels are indicative of a diagnosis of no Alzheimer's Disease in the subject.

The level(s) of the one or more biomarkers may be compared to Alzheimer's Disease-positive and/or Alzheimer's Disease-negative reference levels using various techniques, including a simple comparison (e.g., a manual comparison) of the level(s) of the one or more biomarkers in the biological sample to Alzheimer's Disease-positive and/or Alzheimer's Disease-negative reference levels. The level(s) of the one or more biomarkers in the biological sample may also be compared to Alzheimer's Disease-positive and/or Alzheimer's Disease-negative reference levels using one or more statistical analyses (e.g., t-test, Welch's T-test, Wilcoxon's rank sum test, random forest).

In addition, the biological samples may be analyzed to determine the level(s) of one or more non-biomarker compounds. The level(s) of such non-biomarker compounds may also allow differentiation of Alzheimer's Disease from other neurodegenerative diseases that may have similar or overlapping biomarkers to Alzheimer's Disease (as compared to a subject not having a neurodegenerative disease). For example, a known non-biomarker compound present in biological samples of subjects having Alzheimer's Disease and subjects not having Alzheimer's Disease could be monitored to verify a diagnosis of Alzheimer's Disease as compared to a diagnosis of another neurodegenerative disease when biological samples from subjects having the other neurodegenerative disease do not have the non-biomarker compound.

### III. Methods of Determining Predisposition to Alzheimer's Disease

The identification of biomarkers for Alzheimer's Disease also allows for the determination of whether a subject having no symptoms of Alzheimer's Disease is predisposed to developing Alzheimer's Disease. A method of determining whether a subject having no symptoms of Alzheimer's Disease is predisposed to developing Alzheimer's Disease comprises (1) analyzing a biological sample from a subject to determine the level(s) of one or more biomarkers listed in Tables 1, 2, and/or 3 in the sample and (2) comparing the level(s) of the one or more biomarkers in the sample to Alzheimer's Disease-positive and/or Alzheimer's Disease-negative reference levels of the one or more biomarkers in order to determine whether the subject is predisposed to developing Alzheimer's Disease. The results of the method may be used along with other methods (or the results thereof) useful in the clinical determination of whether a subject is predisposed to developing Alzheimer's Disease.

As described above in connection with methods of diagnosing (or aiding in the diagnosis of) Alzheimer's Disease, any suitable method may be used to analyze the biological sample in order to determine the level(s) of the one or more biomarkers in the sample.

As with the methods of diagnosing (or aiding in the diagnosis of) Alzheimer's Disease described above, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 1, 2, and/or 3 or any fraction thereof, may be determined and used in methods of determining whether a subject having no symptoms of Alzheimer's Disease is predisposed to developing Alzheimer's Disease.

After the level(s) of the one or more biomarkers in the sample are determined, the level(s) are compared to Alzheimer's Disease-positive and/or Alzheimer's Disease-negative reference levels in order to predict whether the subject is predisposed to developing Alzheimer's Disease. Levels of the one or more biomarkers in a sample matching the Alzheimer's Disease-positive reference levels (e.g., levels that are the same as the reference levels, substantially the same as the reference levels, above and/or below the minimum and/or maximum of the reference levels, and/or within the range of the reference levels) are indicative of the subject being predisposed to developing Alzheimer's Disease. Levels of the one or more biomarkers in a sample matching the Alzheimer's Disease-negative reference levels (e.g., levels that are the same as the reference levels, substantially the same as the reference levels, above and/or below the minimum and/or maximum of the reference levels, and/or within the range of the reference levels) are indicative of the subject not being predisposed to developing Alzheimer's Disease. In addition, levels of the one or more biomarkers that are differentially present (especially at a level that is statistically significant) in the sample as compared to Alzheimer's Disease-negative reference levels are indicative of the subject being predisposed to developing Alzheimer's Disease. Levels of the one or more biomarkers that are differentially present (especially at a level that is statistically significant) in the sample as compared to Alzheimer's Disease-positive reference levels are indicative of the subject not being predisposed to developing Alzheimer's Disease.

Furthermore, it may also be possible to determine reference levels specific to assessing whether or not a subject that does not have Alzheimer's Disease is predisposed to developing Alzheimer's Disease. For example, it may be possible to determine reference levels of the biomarkers for assessing different degrees of risk (e.g., low, medium, high) in a subject for developing Alzheimer's Disease. Such reference levels could be used for comparison to the levels of the one or more biomarkers in a biological sample from a subject.

As with the methods described above, the level(s) of the one or more biomarkers may be compared to Alzheimer's Disease-positive and/or Alzheimer's Disease-negative reference levels using various techniques, including a simple comparison, one or more statistical analyses, and combinations thereof.

As with the methods of diagnosing (or aiding in diagnosing) whether a subject has Alzheimer's Disease, the methods of determining whether a subject having no symptoms of Alzheimer's Disease is predisposed to developing Alzheimer's Disease may further comprise analyzing the biological sample to determine the level(s) of one or more non-biomarker compounds.

### IV. Methods of Monitoring Progression/Regression of Alzheimer's Disease

The identification of biomarkers for Alzheimer's Disease also allows for monitoring progression/regression of Alzheimer's Disease in a subject. A method of monitoring the progression/regression of Alzheimer's Disease in a subject comprises (1) analyzing a first biological sample from a subject to determine the level(s) of one or more biomarkers for Alzheimer's Disease selected from Tables 1, 2, and/or 3, the first sample obtained from the subject at a first time point, (2) analyzing a second biological sample from a subject to determine the level(s) of the one or more biomarkers, the second sample obtained from the subject at a second time point, and (3) comparing the level(s) of one or more biomarkers in the first sample to the level(s) of the one or more biomarkers in the second sample in order to monitor the progression/regression of Alzheimer's Disease in the subject. The results of the method are indicative of the course of Alzheimer's Disease (i.e., progression or regression, if any change) in the subject.

The change (if any) in the level(s) of the one or more biomarkers over time may be indicative of progression or regression of Alzheimer's Disease in the subject. In order to characterize the course of Alzheimer's Disease in the subject, the level(s) of the one or more biomarkers in the first sample, the level(s) of the one or more biomarkers in the second sample, and/or the results of the comparison of the levels of the biomarkers in the first and second samples may be compared to Alzheimer's Disease-positive and/or Alzheimer's Disease-negative reference levels of the one or more biomarkers. If the comparisons indicate that the level(s) of the one or more biomarkers are increasing or decreasing over time (e.g., in the second sample as compared to the first sample) to become more similar to the Alzheimer's Disease-positive reference levels (or less similar to the Alzheimer's Disease-negative reference levels), then the results are indicative of Alzheimer's Disease progression. If the comparisons indicate that the level(s) of the one or more biomarkers are increasing or decreasing over time to become more similar to the Alzheimer's Disease-negative reference levels (or less similar to the Alzheimer's Disease-positive reference levels), then the results are indicative of Alzheimer's Disease regression.

As with the other methods described herein, the comparisons made in the methods of monitoring progression/regression of Alzheimer's Disease in a subject may be carried out using various techniques, including simple comparisons, one or more statistical analyses, and combinations thereof.

The results of the method may be used along with other methods (or the results thereof) useful in the clinical monitoring of progression/regression of Alzheimer's Disease in a subject.

As described above in connection with methods of diagnosing (or aiding in the diagnosis of) Alzheimer's Disease, any suitable method may be used to analyze the biological samples in order to determine the level(s) of the one or more biomarkers in the samples. In addition, the level(s) one or more biomarkers, including a combination of all of the biomarkers in Tables 1, 2, and/or 3 or any fraction thereof, may be determined and used in methods of monitoring progression/regression of Alzheimer's Disease in a subject.

Such methods could be conducted to monitor the course of Alzheimer's Disease in subjects having Alzheimer's Disease or could be used in subjects not having Alzheimer's Disease (e.g., subjects suspected of being predisposed to developing Alzheimer's Disease) in order to monitor levels of predisposition to Alzheimer's Disease.

### V. Methods of Assessing Efficacy of Compositions for Treating Alzheimer's Disease

The identification of biomarkers for Alzheimer's Disease also allows for assessment of the efficacy of a composition for treating Alzheimer's Disease as well as the assessment of the relative efficacy of two or more compositions for treating Alzheimer's Disease. Such assessments may be used, for example, in efficacy studies as well as in lead selection of compositions for treating Alzheimer's Disease.

A method of assessing the efficacy of a composition for treating Alzheimer's Disease comprises (1) analyzing, from a subject (or a group of subjects) having Alzheimer's Disease and currently or previously being treated with a composition, a biological sample (or a group of samples) to determine the level(s) of one or more biomarkers selected from Tables 1, 2, and/or 3 and (2) comparing the level(s) of the one or more biomarkers in the sample(s) to (a) level(s) of the one or more biomarkers in a previously-taken biological sample from the subject, wherein the previously-taken biological sample was obtained from the subject before being treated with the composition, (b) Alzheimer's Disease-positive reference levels of the one or more biomarkers, and/or (c) Alzheimer's Disease-negative reference levels of the one or more biomarkers. The results of the comparison are indicative of the efficacy of the composition for treating Alzheimer's Disease.

Thus, in order to characterize the efficacy of the composition for treating Alzheimer's Disease, the level(s) of the one or more biomarkers in the biological sample are compared to (1) Alzheimer's Disease-positive reference levels, (2) Alzheimer's Disease-negative reference levels, and/or (3) previous levels of the one or more biomarkers in the subject before treatment with the composition.

When comparing the level(s) of the one or more biomarkers in the biological sample (from a subject having Alzheimer's Disease and currently or previously being treated with a composition) to Alzheimer's Disease-positive reference levels and/or Alzheimer's Disease-negative reference levels, level(s) in the sample matching the Alzheimer's Disease-negative reference levels (e.g., levels that are the same as the reference levels, substantially the same as the reference levels, above and/or below the minimum and/or maximum of the reference levels, and/or within the range of the reference levels) are indicative of the composition having efficacy for treating Alzheimer's Disease. Levels of the one or more biomarkers in the sample matching the Alzheimer's Disease-positive reference levels (e.g., levels that are the same as the reference levels, substantially the same as the reference levels, above and/or below the minimum and/or maximum of the reference levels, and/or within the range of the reference levels) are indicative of the composition not having efficacy for treating Alzheimer's Disease. The comparisons may also indicate degrees of efficacy for treating Alzheimer's Disease based on the level(s) of the one or more biomarkers.

When the level(s) of the one or more biomarkers in the biological sample (from a subject having Alzheimer's Disease and currently or previously being treated with a composition) are compared to level(s) of the one or more biomarkers in a previously-taken biological sample from the subject before treatment with the composition, any changes in the level(s) of the one or more biomarkers are indicative of the efficacy of the composition for treating Alzheimer's Disease. That is, if the comparisons indicate that the level(s) of the one or more biomarkers have increased or decreased after treatment with the composition to become more similar to the Alzheimer's Disease-negative reference levels (or less similar to the Alzheimer's Disease-positive reference levels), then the results are indicative of the composition having efficacy for treating Alzheimer's Disease. If the comparisons indicate that the level(s) of the one or more biomarkers have not increased or decreased after treatment with the composition to become more similar to the Alzheimer's Disease-negative reference levels (or less similar to the Alzheimer's Disease-positive reference levels), then the results are indicative of the composition not having efficacy for treating Alzheimer's Disease. The comparisons may also indicate degrees of efficacy for treating Alzheimer's Disease based on the amount of changes observed in the level(s) of the one or more biomarkers after treatment. In order to help characterize such a comparison, the changes in the level(s) of the one or more biomarkers, the level(s) of the one or more biomarkers before treatment, and/or the level(s) of the one or more biomarkers in the subject currently or previously being treated with the composition may be compared to Alzheimer's Disease-positive and/or Alzheimer's Disease-negative reference levels of the one or more biomarkers.

Another method for assessing the efficacy of a composition in treating Alzheimer's Disease (AD) comprises (1) analyzing a first biological sample from a subject to determine the level(s) of one or more biomarkers selected from Tables 1, 2, and/or 3, the first sample obtained from the subject at a first time point, (2) administering the composition to the subject, (3) analyzing a second biological sample from a subject to determine the level(s) of the one or more biomarkers, the second sample obtained from the subject at a second time point after administration of the composition, and (4) comparing the level(s) of one or more biomarkers in the first sample to the level(s) of the one or more biomarkers in the second sample in order to assess the efficacy of the composition for treating Alzheimer's Disease. As indicated above, if the comparison of the samples indicates that the level(s) of the one or more biomarkers have increased or decreased after administration of the composition to become more similar to the Alzheimer's Disease-negative reference levels (or less similar to the Alzheimer's Disease-positive reference levels), then the results are indicative of the composition having efficacy for treating Alzheimer's Disease. If the comparison indicates that the level(s) of the one or more biomarkers have not increased or decreased after administration of the composition to become more similar to the Alzheimer's Disease-negative reference levels (or less similar to the Alzheimer's Disease-positive reference levels), then the results are indicative of the composition not having efficacy for treating Alzheimer's Disease. The comparison may also indicate a degree of efficacy for treating Alzheimer's Disease based on the amount of changes observed in the level(s) of the one or more biomarkers after administration of the composition. In order to help characterize such a comparison, the changes in the level(s) of the one or more biomarkers, the level(s) of the one or more biomarkers before administration of the composition, and/or the level(s) of the one or more biomarkers after administration of the composition may be compared to Alzheimer's Disease-positive and/or Alzheimer's Disease-negative reference levels of the one or more biomarkers of the two compositions.

A method of assessing the relative efficacy of two or more compositions for treating Alzheimer's Disease comprises (1) analyzing, from a first subject having Alzheimer's Disease and currently or previously being treated with a first composition, a first biological sample to determine the level(s) of one or more biomarkers selected from Tables 1, 2, and/or 3, (2) analyzing, from a second subject having Alzheimer's Disease and currently or previously being treated with a second composition, a second biological sample to determine the level(s) of the one or more biomarkers, and (3) comparing the level(s) of one or more biomarkers in the first sample to the level(s) of the one or more biomarkers in the second sample in order to assess the relative efficacy of the first and second compositions for treating Alzheimer's Disease. The results are indicative of the relative efficacy of the two compositions, and the results (or the levels of the one or more biomarkers in the first sample and/or the level(s) of the one or more biomarkers in the second sample) may be compared to Alzheimer's Disease-positive or Alzheimer's Disease-negative reference levels to aid in characterizing the relative efficacy.

Each of the methods of assessing efficacy may be conducted on one or more subjects or one or more groups of subjects (e.g., a first group being treated with a first composition and a second group being treated with a second composition).

As with the other methods described herein, the comparisons made in the methods of assessing efficacy (or relative efficacy) of compositions for treating Alzheimer's Disease may be carried out using various techniques, including simple comparisons, one or more statistical analyses, and combinations thereof. Any suitable method may be used to analyze the biological samples in order to determine the level(s) of the one or more biomarkers in the samples. In addition, the level(s) of one or more biomarkers, including a combination of all of the biomarkers in Tables 1, 2, and/or 3 or any fraction thereof, may be determined and used in methods of assessing efficacy (or relative efficacy) of compositions for treating Alzheimer's Disease.

Finally, the methods of assessing efficacy (or relative efficacy) of one or more compositions for treating Alzheimer's Disease may further comprise analyzing the biological sample to determine the level(s) of one or more non-biomarker compounds. The non-biomarker compounds may then be compared to reference levels of non-biomarker compounds for subjects having (or not having) Alzheimer's Disease.

VI. Methods of Screening a Composition for Activity in Modulating Biomarkers Associated with Alzheimer's Disease

The identification of biomarkers for Alzheimer's Disease also allows for the screening of compositions for activity in modulating biomarkers associated with Alzheimer's Disease, which may be useful in treating Alzheimer's Disease. Methods of screening compositions useful for treatment of Alzheimer's Disease comprise assaying test compositions for activity in modulating the levels of one or more biomarkers in Tables 1, 2, and/or 3. Such screening assays may be conducted in vitro and/or in vivo, and may be in any form known in the art useful for assaying modulation of such biomarkers in the presence of a test composition such as, for example, cell culture assays, organ culture assays, and in vivo assays (e.g., assays involving animal models).

In one embodiment, a method for screening a composition for activity in modulating one or more biomarkers of Alzheimer's Disease comprises (1) contacting one or more cells with a composition, (2) analyzing at least a portion of the one or more cells or a biological sample associated with the cells to determine the level(s) of one or more biomarkers of Alzheimer's Disease selected from Tables 1, 2, and/or 3; and (3) comparing the level(s) of the one or more biomarkers with predetermined standard levels for the one or more biomarkers to determine whether the composition modulated the level(s) of the one or more biomarkers. As discussed above, the cells may be contacted with the composition in vitro and/or in vivo. The predetermined standard levels for the one or more biomarkers may be the levels of the one or more biomarkers in the one or more cells in the absence of the composition. The predetermined standard levels for the one or more biomarkers may also be the level(s) of the one or more biomarkers in control cells not contacted with the composition.

In addition, the methods may further comprise analyzing at least a portion of the one or more cells or a biological sample associated with the cells to determine the level(s) of one or more non-biomarker compounds of Alzheimer's Disease. The levels of the non-biomarker compounds may then be compared to predetermined standard levels of the one or more non-biomarker compounds.

Any suitable method may be used to analyze at least a portion of the one or more cells or a biological sample associated with the cells in order to determine the level(s) of the one or more biomarkers (or levels of non-biomarker compounds). Suitable methods include chromatography (e.g., HPLC, gas chromatograph, liquid chromatography), mass spectrometry (e.g., MS, MS-MS), ELISA, antibody linkage, other immunochemical techniques, and combinations thereof. Further, the level(s) of the one or more biomarkers (or levels of non-biomarker compounds) may be measured indirectly, for example, by using an assay that measures the level of a compound (or compounds) that correlates with the level of the biomarker(s) (or non-biomarker compounds) that are desired to be measured.

### VII. Method of Identifying Potential Drug Targets

The identification of biomarkers for Alzheimer's Disease also allows for the identification of potential drug targets for Alzheimer's Disease. A method for identifying a potential drug target for Alzheimer's Disease (AD) comprises (1) identifying one or more biochemical pathways associated with one or more biomarkers for Alzheimer's Disease selected from Tables 1, 2, and/or 3 and (2) identifying a protein (e.g., an enzyme) affecting at least one of the one or more identified biochemical pathways, the protein being a potential drug target for Alzheimer's Disease.

Another method for identifying a potential drug target for Alzheimer's Disease (AD) comprises (1) identifying one or more biochemical pathways associated with one or more biomarkers for Alzheimer's Disease selected from Tables 1, 2, and/or 3 and one or more non-biomarker compounds of Alzheimer's Disease selected from Tables 1, 2, and/or 3 and (2) identifying a protein affecting at least one of the one or more identified biochemical pathways, the protein being a potential drug target for Alzheimer's Disease.

One or more biochemical pathways (e.g., biosynthetic and/or metabolic (catabolic) pathway) are identified that are associated with one or more biomarkers (or non-biomarker compounds). After the biochemical pathways are identified, one or more proteins affecting at least one of the pathways are identified. Preferably, those proteins affecting more than one of the pathways are identified.

A build-up of one metabolite (e.g., a pathway intermediate) may indicate the presence of a ‘block’ downstream of the metabolite and the block may result in a low/absent level of a downstream metabolite (e.g. product of a biosynthetic pathway). In a similar manner, the absence of a metabolite could indicate the presence of a ‘block’ in the pathway upstream of the metabolite resulting from inactive or non-functional enzyme(s) or from unavailability of biochemical intermediates that are required substrates to produce the product. Alternatively, an increase in the level of a metabolite could indicate a genetic mutation that produces an aberrant protein which results in the over-production and/or accumulation of a metabolite which then leads to an alteration of other related biochemical pathways and result in dysregulation of the normal flux through the pathway; further, the build-up of the biochemical intermediate metabolite may be toxic or may compromise the production of a necessary intermediate for a related pathway. It is possible that the relationship between pathways is currently unknown and this data could reveal such a relationship.

The proteins identified as potential drug targets may then be used to identify compositions that may be potential candidates for treating Alzheimer's Disease, including compositions for gene therapy.

### VIII. Methods of Treating Alzheimer's Disease

The identification of biomarkers for Alzheimer's Disease also allows for the treatment of Alzheimer's Disease. For example, in order to treat a subject having Alzheimer's Disease, an effective amount of one or more Alzheimer's Disease biomarkers that are lowered in Alzheimer's Disease as compared to a healthy subject not having Alzheimer's Disease may be administered to the subject. The biomarkers that may be administered may comprise one or more of the biomarkers in Tables 1, 2, and/or 3 that are decreased in Alzheimer's Disease. Such biomarkers could be isolated based on the analytical characterizations for the biomarkers listed in Tables 1, 2, and/or 3. In some embodiments, the biomarkers that are administered are one or more biomarkers listed in Tables 1, 2, and/or 3 that are decreased in Alzheimer's Disease and that have a p-value less than 0.05 and/or a q-value of less than 0.10. In other embodiments, the biomarkers that are administered are one or biomarkers listed in Tables 1, 2, and/or 3 that are decreased in Alzheimer's Disease by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent).

### IX. Methods of Using the Alzheimer's Disease Biomarkers for Other Neurodegenerative Diseases

It is believed that some of the biomarkers for Alzheimer's Disease described herein may also be biomarkers for neurodegenerative diseases in general. Therefore, it is believed that at least some of the Alzheimer's Disease biomarkers may be used in the methods described herein for neurodegenerative diseases in general. That is, the methods described herein with respect to Alzheimer's Disease may also be used for diagnosing (or aiding in the diagnosis of) a neurodegenerative disease, methods of monitoring progression/regression of a neurodegenerative disease, methods of assessing efficacy of compositions for treating a neurodegenerative disease, methods of screening a composition for activity in modulating biomarkers associated with a neurodegenerative disease, methods of identifying potential drug targets for neurodegenerative diseases, and methods of treating a neurodegenerative disease. Such methods could be conducted as described herein with respect to Alzheimer's Disease.

### X. Other Methods

Other methods of using the biomarkers discussed herein are also contemplated. For example, the methods described in U.S. Pat. No. 7,005,255 and U.S. patent application Ser. Nos. 11/357,732, 10/695,265, (Publication No. 2005/0014132), Ser. No. 11/301,077 (Publication No. 2006/0134676), Ser. No. 11/301,078 (Publication No. 2006/0134677), Ser. No. 11/301,079 (Publication No. 2006/0134678), and Ser. No. 11/405,033, may be conducted using a small molecule profile comprising one or more of the biomarkers disclosed herein and/or one or more of the non-biomarker compounds disclosed herein.

In any of the methods listed herein, the biomarkers that are used may be selected from those biomarkers in Tables 1, 2, and/or 3 having p-values of less than 0.05 and/or those biomarkers in Tables 1, 2, and/or 3 having q-values of less than 0.10. The biomarkers that are used in any of the methods described herein may also be selected from those biomarkers in Tables 1, 2, and/or 3 that are decreased in Alzheimer's Disease (as compared to the control) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); and/or those biomarkers in Tables 1, 2, and/or 3 that are increased in Alzheimer's Disease (as compared to the control) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more.

### EXAMPLES

The invention will be further explained by the following illustrative examples that are intended to be non-limiting.

### I. General Methods:

A. Identification of Metabolic Profiles for Alzheimer's Disease

Each sample was analyzed to determine the concentration of several hundred metabolites. Analytical techniques such as GC-MS (gas chromatography-mass spectrometry) and LC-MS (liquid chromatography-mass spectrometry) were used to analyze the metabolites. Multiple aliquots were simultaneously, and in parallel, analyzed, and, after appropriate quality control (QC), the information derived from each analysis was recombined. Every sample was characterized according to several thousand characteristics, which ultimately amount to several hundred chemical species. The techniques used were able to identify novel and chemically unnamed compounds.

B. Statistical Analysis

The data was analyzed using several statistical methods to identify molecules (either known, named metabolites or unnamed metabolites) present at differential levels in a definable population or subpopulation (e.g., biomarkers for Alzheimer's Disease biological samples compared to control biological samples) useful for distinguishing between the definable populations (e.g., Alzheimer's Disease and control). Other molecules (either known, named metabolites or unnamed metabolites) in the definable population or subpopulation were also identified.

Two types of statistical analysis were performed: (1) significance tests and (2) classification analysis:
(1) For pair-wise comparisons, Welch's t-tests and/or Wilcoxon's rank sum tests were performed. For other statistical designs various ANOVA procedures (e.g., repeated measures ANOVA) were perfumed.
(2) For classification, random forest analyses was used. Random forests give an estimate of how well individuals in a new data set can be classified into existing groups, in contrast to a t-test, which tests whether the unknown mean values for two populations are different or not. Random forests create a set of classification trees based on continual sampling of the experimental units and compounds. Then each observation is classified based on the majority votes from all the classification trees.

Statistical analyses were performed with the program “R” available on the worldwide web at the website cran.r-project.org/.

C. Biomarker Identification

Various peaks identified in the analyses (e.g. GC-MS, LC-MS, MS-MS), including those identified as statistically significant, were subjected to a mass spectrometry based chemical identification process.

### Example 1

### Identification of Biomarkers that Distinguish Late Stage AD from Healthy Controls

In one example, biomarkers were discovered by (1) analyzing plasma samples from different groups of human subjects to determine the levels of metabolites in the samples and then (2) statistically analyzing the results to determine those metabolites that are differentially present in the two groups. The plasma samples used for the analysis were from Late Stage Alzheimer's Disease subjects and control subjects not diagnosed with Alzheimer's Disease. Late stage AD was determined by looking at the Right Blessed Dementia Score (RBDS). The RBDS is a scale from 0-37 in a cognitive test. More than 3 mistakes in this test indicate cognitive impairment. When the patient is in advanced stages of dementia and cannot be tested, they are given a score of −50. All of the late stage AD patients were used to calculate the values in Table 1 had a RBDS of −50.

After the levels of metabolites were determined, the data was analyzed using T-tests (Table 1).

### Biomarkers

As listed below in Table 1, biomarkers were discovered that were differentially present between samples from Late Stage Alzheimer's Disease subjects and Control subjects not diagnosed with Alzheimer's Disease.

Table 1 includes, for each listed biomarker compound, the p-value and the q-value determined in the statistical analysis of the data concerning the biomarkers, an indication of whether the mean level of a particular compound was higher in the Alzheimer's Disease or Control samples (a “+” indicating a higher mean level in Alzheimer's Disease samples as compared to the control samples and a “−” indicating a lower mean level in Alzheimer's Disease samples as compared to the control samples), and an indication of the percentage difference in the Alzheimer's Disease mean level as compared to the control mean level. Throughout the tables, names of metabolites ending with the notation “−35” indicate that the levels of those compounds were measured using LC-MS, and names ending with the notation “−9” indicate that the levels of those compounds were measured using GC-MS. The term “Isobar” as used in the table indicates the compounds that could not be distinguished from each other on the analytical platform used in the analysis (i.e., the compounds in an isobar elute at nearly the same time and have similar (and sometimes exactly the same) quant ions, and thus cannot be distinguished).

Non-biomarker compounds identified in the analysis are also listed in Table 1 below as those compounds that have a percentage change in Alzheimer's Disease of 0%.

TABLE 1
Control vs Late Stage AD (−50 by RBDS)

| | | | Increase | % |
|---|---|---|---|---|
| | | | (+) or | Change |
| | | | Decrease | in |
| Compound | p-value | q-value | (−) In AD | AD |
| Hippuric acid-35 | <0.0001 | <0.0001 | − | −283% |
| Metabolite-3055-35 | <0.0001 | <0.0001 | − | −326% |
| Metabolite-3604-35 | <0.0001 | 0.0001 | − | −315% |
| Metabolite-2270-35 | 0.0001 | 0.0063 | − | −487% |
| Metabolite-3765-35 | 0.0001 | 0.0073 | − | −213% |
| Metabolite-2005-35 | 0.0003 | 0.0171 | − | −99% |
| Metabolite-2250-35 | 0.0004 | 0.0174 | − | −153% |
| Metabolite-3327-35 | 0.0006 | 0.0238 | − | −254% |
| Metabolite-1193-35 | 0.0013 | 0.0478 | − | −91% |
| Metabolite-1116-35 | 0.0016 | 0.0521 | − | −33% |
| Metabolite-3218-35 | 0.0017 | 0.0521 | − | −44% |
| Metabolite-4003-35 | 0.0021 | 0.0604 | − | −111% |
| Metabolite-1342-35 | 0.0025 | 0.0617 | − | −162% |
| Metabolite-3762-35 | 0.0025 | 0.0617 | − | −45% |
| Metabolite-3072-35 | 0.0042 | 0.0866 | − | −139% |
| Metabolite-1323-possible-4- | 0.0043 | 0.0866 | − | −115% |
| sulfobenzyl-alcohol-35 | | | | |
| Metabolite-2348-35 | 0.0043 | 0.0866 | − | −127% |
| Metabolite-614-9 | 0.0046 | 0.0877 | − | −52% |
| Metabolite-2185-35 | 0.0049 | 0.0896 | − | −64% |
| Metabolite-1465-35 | 0.0052 | 0.0896 | − | −59% |
| Metabolite-2291-35 | 0.0063 | 0.1019 | − | −230% |
| Metabolite-1802-35 | 0.0064 | 0.1019 | − | −75% |
| pantothenic acid-35 | 0.0071 | 0.1084 | − | −75% |
| Metabolite-3739-35 | 0.0093 | 0.1334 | − | −116% |
| Metabolite-2258-35 | 0.0096 | 0.1334 | − | −68% |
| cholesterol-9 | 0.0098 | 0.1334 | + | 9% |
| Metabolite-3139-35 | 0.01 | 0.1334 | − | −15% |
| 4-Guanidinobutanoic acid-35 | 0.011 | 0.1351 | − | −21% |
| tartaric acid-35 | 0.0111 | 0.1351 | − | −70% |
| Metabolite-1987-9 | 0.0112 | 0.1351 | + | 8% |
| 3-hydroxybutanoic acid-9 | 0.0116 | 0.1362 | − | −84% |
| Metabolite-2821-35 | 0.014 | 0.1598 | − | −45% |
| Metabolite-1974-35 | 0.0152 | 0.1611 | − | −134% |
| Metabolite-1981-35 | 0.0152 | 0.1611 | − | −109% |
| Metabolite-1911-35 | 0.0153 | 0.1611 | − | −118% |
| Metabolite-1926-35 | 0.0163 | 0.1643 | − | −130% |
| Metabolite-1284-35 | 0.0165 | 0.1643 | − | −23% |
| pyridoxamine-35 | 0.0176 | 0.1715 | − | −27% |
| Metabolite-2567-35 | 0.0194 | 0.1847 | − | −54% |
| Metabolite-2273-35 | 0.0201 | 0.1867 | − | −90% |
| L-kynurenine-35 | 0.0211 | 0.1917 | − | −33% |
| Metabolite-3130-35 | 0.0218 | 0.1936 | − | −50% |
| Metabolite-2558-35 | 0.0241 | 0.2048 | − | −106% |
| 3-amino-isobutyrate-9 | 0.0255 | 0.2109 | + | 31% |
| isocitrate-35 | 0.0263 | 0.2109 | − | −40% |
| Metabolite-1655-35 | 0.0265 | 0.2109 | + | 18% |
| Metabolite-2139-35 | 0.0278 | 0.2109 | − | −42% |
| Metabolite-1498-35 | 0.0279 | 0.2109 | − | −36% |
| Metabolite-1914-35 | 0.028 | 0.2109 | − | −127% |
| oxitryptan-35 | 0.0306 | 0.2266 | − | −59% |
| L-alpha- | 0.0324 | 0.2355 | + | 52% |
| glycerophosphorylcholine-35 | | | | |
| possible-L-Alanine-d7-9 | 0.0422 | 0.2983 | + | 9% |
| Metabolite-670-9 | 0.0426 | 0.2983 | + | 11% |
| Metabolite-3816-35 | 0.0449 | 0.3089 | − | −21% |
| glutamic acid-9 | 0.0482 | 0.3261 | − | −57% |
| Metabolite-3044-35 | 0.0493 | 0.3281 | − | −20% |
| lactate-9 | 0.0509 | 0.3331 | + | 14% |
| histidine-35 | 0.0521 | 0.3358 | − | −27% |
| Metabolite-261-9 | 0.0545 | 0.3453 | − | −34% |
| Metabolite-3282-9 | 0.0576 | 0.3524 | + | 34% |
| Metabolite-2894-35 | 0.0582 | 0.3524 | − | −75% |
| Metabolite-1988-35 | 0.0595 | 0.3524 | − | −78% |
| Metabolite-407-9 | 0.0603 | 0.3524 | − | −21% |
| Metabolite-2722-9 | 0.0607 | 0.3524 | + | 6% |
| Metabolite-3181-35 | 0.0609 | 0.3524 | − | −58% |
| Metabolite-1110-35 | 0.0638 | 0.3607 | − | −93% |
| Metabolite-451-9 | 0.0641 | 0.3607 | + | 16% |
| Metabolite-3781-35 | 0.0669 | 0.367 | − | −18% |
| alpha-tocopherol-9 | 0.068 | 0.367 | + | 66% |
| Metabolite-3651-35 | 0.0692 | 0.367 | − | −5% |
| Metabolite-3131-35 | 0.0697 | 0.367 | − | −44% |
| Metabolite-3733-35 | 0.0699 | 0.367 | − | −41% |
| Metabolite-1368-35 | 0.0728 | 0.3732 | − | −200% |
| Metabolite-3183-35 | 0.0739 | 0.3732 | − | −30% |
| Metabolite-3313-35 | 0.0748 | 0.3732 | − | −42% |
| Metabolite-3956-35 | 0.0818 | 0.3958 | − | −85% |
| Metabolite-2806-35 | 0.0831 | 0.3958 | − | −20% |
| Metabolite-3413-9 | 0.0834 | 0.3958 | + | 6% |
| Metabolite-669-9 | 0.0854 | 0.3958 | + | 33% |
| gluconic acid-9 | 0.0865 | 0.3958 | + | 9% |
| Metabolite-1828-9 | 0.0881 | 0.3958 | + | 30% |
| Metabolite-3134-35 | 0.0882 | 0.3958 | − | −85% |
| Metabolite-3810-35 | 0.0939 | 0.4066 | − | −90% |
| N-carbamoyl-L-aspartate-35 | 0.0944 | 0.4066 | − | −50% |
| Metabolite-1866-9 | 0.0948 | 0.4066 | + | 27% |
| possible-TMS-2-t-butyl-6- | 0.0956 | 0.4066 | + | 23% |
| methylphenol-9 | | | | |
| Metabolite-2074-35 | 0.0957 | 0.4066 | − | −45% |
| Metabolite-470-9 | 0.0987 | 0.415 | + | 12% |
| Metabolite-3766-35 | 0.1024 | 0.4222 | − | −50% |
| Metabolite-3657-35 | 0.103 | 0.4222 | − | −6% |
| Metabolite-3760-35 | 0.1036 | 0.4222 | − | −20% |
| Metabolite-3905-9 | 0.1067 | 0.4297 | + | 9% |
| Metabolite-3633-9 | 0.1076 | 0.4297 | + | 8% |
| Metabolite-3138-35 | 0.11 | 0.4337 | − | −22% |
| Metabolite-1345-35 | 0.1108 | 0.4337 | − | −59% |
| Metabolite-578-9 | 0.1187 | 0.4511 | + | 16% |
| Metabolite-2693-35 | 0.1191 | 0.4511 | + | 41% |
| tetradecanoic acid-9 | 0.1196 | 0.4511 | − | −27% |
| methyl-indole-3-acetate-35 | 0.1197 | 0.4511 | − | −11% |
| Metabolite-3252-9 | 0.1217 | 0.453 | + | 6% |
| Metabolite-1069-35 | 0.1234 | 0.453 | − | −98% |
| Metabolite-268-9 | 0.1245 | 0.453 | − | −14% |
| possible-arabinose-3-9 | 0.1248 | 0.453 | + | 22% |
| creatinine-35 | 0.126 | 0.4532 | − | −12% |
| Metabolite-2041-35 | 0.1273 | 0.4538 | − | −24% |
| Metabolite-543-9 | 0.13 | 0.4569 | − | −51% |
| Metabolite-3370-35 | 0.1305 | 0.4569 | − | −25% |
| Metabolite-2285-35 | 0.1316 | 0.4569 | − | −38% |
| Metabolite-3664-35 | 0.1342 | 0.4611 | + | 17% |
| Metabolite-785-9 | 0.1353 | 0.4611 | + | 6% |
| Metabolite-3754-35 | 0.1362 | 0.4611 | − | −21% |
| Metabolite-3211-9 | 0.1386 | 0.4652 | + | 18% |
| Metabolite-1289-35 | 0.1406 | 0.468 | − | −22% |
| Metabolite-1956-probable- | 0.1437 | 0.4742 | + | 28% |
| amino-malonic acid-NIST-9 | | | | |
| gamma-L-glutamyl-L-tyrosine- | 0.147 | 0.4812 | − | −20% |
| 35 | | | | |
| Metabolite-3705-35 | 0.1493 | 0.4816 | − | −23% |
| Metabolite-1111-35 | 0.1525 | 0.4816 | − | −19% |
| sn-Glycerol-3-phosphate-35 | 0.1529 | 0.4816 | − | −7% |
| Metabolite-2390-35 | 0.1533 | 0.4816 | − | −29% |
| sucrose-9 | 0.154 | 0.4816 | − | −147% |
| phosphate-9 | 0.1551 | 0.4816 | + | 9% |
| Metabolite-3843-35 | 0.1595 | 0.4862 | − | −12% |
| possible-Salicyluric acid-tris- | 0.1609 | 0.4868 | + | 6% |
| TMS-9 | | | | |
| DL-pipecolic acid-35 | 0.1746 | 0.5203 | − | −19% |
| urea2-9 | 0.1776 | 0.5254 | − | −10% |
| Metabolite-1576-35 | 0.1802 | 0.5262 | − | −17% |
| Metabolite-4114-35 | 0.1855 | 0.5332 | − | −37% |
| Metabolite-2113-9 | 0.1856 | 0.5332 | + | 28% |
| Metabolite-2269-35 | 0.1928 | 0.5445 | − | −70% |
| Metabolite-3170-9 | 0.1947 | 0.5445 | − | −18% |
| Metabolite-3165-35 | 0.195 | 0.5445 | − | −11% |
| Metabolite-3708-35 | 0.2047 | 0.5622 | − | −20% |
| alpha-Hydroxyisobutyric acid- | 0.2063 | 0.5622 | − | −18% |
| tms-9 | | | | |
| Metabolite-2220-TMS- | 0.2084 | 0.5622 | − | −59% |
| pyrophosphate-9 | | | | |
| possible-2-hydroxypropanoic | 0.2125 | 0.5628 | + | 18% |
| acid-lactate-high-concentration-9 | | | | |
| Metabolite-3326-35 | 0.2141 | 0.5628 | − | −27% |
| Metabolite-2008-35 | 0.2143 | 0.5628 | − | −27% |
| Metabolite-3667-35 | 0.2147 | 0.5628 | + | 21% |
| Metabolite-2173-35 | 0.2169 | 0.5628 | − | −23% |
| Metabolite-565-9 | 0.2178 | 0.5628 | + | 3% |
| Metabolite-2760-9 | 0.2192 | 0.5628 | + | 18% |
| Metabolite-3230-35 | 0.2199 | 0.5628 | − | −14% |
| alpha-D-ribose-5-phosphate-9 | 0.2226 | 0.5661 | − | −16% |
| Metabolite-1656-35 | 0.2243 | 0.5668 | − | −44% |
| Metabolite-3692-35 | 0.2281 | 0.5729 | − | −12% |
| Metabolite-485-9 | 0.2338 | 0.5799 | + | 24% |
| possible-D-fructose-9 | 0.2369 | 0.5802 | + | 56% |
| Metabolite-3710-35 | 0.2379 | 0.5802 | + | 81% |
| Metabolite-2895-35 | 0.2383 | 0.5802 | − | −102% |
| glutarate-9 | 0.247 | 0.5882 | + | 34% |
| Metabolite-2320-35 | 0.2472 | 0.5882 | − | −24% |
| Metabolite-2790-9 | 0.2486 | 0.5882 | − | −8% |
| Metabolite-3178-35 | 0.2488 | 0.5882 | − | −7% |
| Metabolite-1286-35 | 0.2489 | 0.5882 | − | −12% |
| vitamin-B6-9 | 0.2549 | 0.5903 | + | 6% |
| Metabolite-3665-35 | 0.255 | 0.5903 | + | 7% |
| Metabolite-3797-9 | 0.2557 | 0.5903 | + | 15% |
| Metabolite-3334-35 | 0.2574 | 0.5907 | − | −13% |
| 12-hydroxydodecanoic acid-9 | 0.2607 | 0.5921 | + | 19% |
| Metabolite-1113-35 | 0.2615 | 0.5921 | − | −14% |
| Metabolite-1216-35 | 0.2624 | 0.5921 | + | 13% |
| Metabolite-2753-35 | 0.2659 | 0.5965 | + | 24% |
| Isobar-2-includes-3-amino- | 0.2694 | 0.5981 | − | −13% |
| isobutyrate-2-amino-butyrate-4- | | | | |
| aminobutanoic acid- | | | | |
| dimethylglycine-choline-35 | | | | |
| malic acid-9 | 0.2713 | 0.5981 | + | 16% |
| Metabolite-2322-35 | 0.273 | 0.5981 | − | −36% |
| Metabolite-2774-35 | 0.275 | 0.5981 | − | −36% |
| Metabolite-2548-35 | 0.2777 | 0.5981 | + | 29% |
| Metabolite-2386-35 | 0.2786 | 0.5981 | − | −18% |
| Metabolite-1350-35 | 0.2804 | 0.5981 | + | 44% |
| Metabolite-2306-35 | 0.2816 | 0.5981 | − | −4% |
| Metabolite-2526-35 | 0.2828 | 0.5981 | − | −7% |
| Metabolite-3474-35 | 0.2829 | 0.5981 | − | −24% |
| palmitate-9 | 0.2852 | 0.5981 | − | −14% |
| thyroxine-35 | 0.2856 | 0.5981 | + | 33% |
| Metabolite-3275-9 | 0.2861 | 0.5981 | − | −18% |
| biliverdin-35 | 0.2964 | 0.6065 | − | −27% |
| Metabolite-3052-35 | 0.2964 | 0.6065 | + | 16% |
| Metabolite-2426-35 | 0.3012 | 0.6065 | − | −20% |
| Metabolite-3707-35 | 0.3018 | 0.6065 | − | −70% |
| Isobar-9-includes-sucrose-beta- | 0.3023 | 0.6065 | + | 24% |
| D-lactose-D-trehalose-D- | | | | |
| cellobiose-D-Maltose-palatinose- | | | | |
| melibiose-alpha-D-lactose-35 | | | | |
| Metabolite-3992-35 | 0.3027 | 0.6065 | − | −24% |
| Metabolite-594-9 | 0.3036 | 0.6065 | − | −2% |
| Metabolite-3698-35 | 0.305 | 0.6065 | − | −24% |
| Metabolite-1846-9 | 0.3051 | 0.6065 | − | −9% |
| arginino-succinate-35 | 0.3053 | 0.6065 | + | 16% |
| mannose-6-phosphate-9 | 0.3085 | 0.6098 | + | 14% |
| tyrosine-35 | 0.3184 | 0.624 | − | −13% |
| trans-4-hydroxyproline-35 | 0.3196 | 0.624 | + | 26% |
| Metabolite-3312-35 | 0.3222 | 0.624 | − | −25% |
| Metabolite-3813-35 | 0.3228 | 0.624 | + | 21% |
| Isobar-1-includes-mannose- | 0.3234 | 0.624 | − | −11% |
| fructose-glucose-galactose- | | | | |
| alpha-L-sorbopyranose-Inositol- | | | | |
| D-allose-35 | | | | |
| Isobar-3-methyl-2-oxobutanoate- | 0.3264 | 0.6267 | − | −12% |
| fumaric acid-35 | | | | |
| Metabolite-3475-35 | 0.3305 | 0.6287 | − | −23% |
| Metabolite-3658-35 | 0.3306 | 0.6287 | − | −5% |
| Metabolite-287-9 | 0.3344 | 0.6315 | + | 12% |
| Metabolite-3752-35 | 0.3353 | 0.6315 | − | −59% |
| 3-methyl-2-oxovaleric acid-35 | 0.3407 | 0.6316 | + | 17% |
| 5-oxoproline-35 | 0.3417 | 0.6316 | + | 10% |
| Metabolite-1826-9 | 0.3425 | 0.6316 | + | 20% |
| glycerate-9 | 0.3435 | 0.6316 | − | −13% |
| Metabolite-1346-35 | 0.3471 | 0.6329 | + | 6% |
| Metabolite-1414-9 | 0.3518 | 0.6335 | + | 17% |
| Metabolite-2313-35 | 0.3532 | 0.6335 | − | −18% |
| Metabolite-1331-35 | 0.3534 | 0.6335 | − | −59% |
| Metabolite-1957-9 | 0.355 | 0.6335 | − | −24% |
| Phenylalanine-35 | 0.3567 | 0.6335 | − | −9% |
| Metabolite-3469-35 | 0.357 | 0.6335 | − | −24% |
| Metabolite-3794-9 | 0.36 | 0.6345 | + | 5% |
| Metabolite-3738-35 | 0.3623 | 0.6345 | − | −13% |
| Metabolite-2561-35 | 0.3633 | 0.6345 | − | −29% |
| DOPA-9 | 0.3645 | 0.6345 | − | −20% |
| Glycerol-9 | 0.3673 | 0.6345 | + | 14% |
| Glycine-9 | 0.3676 | 0.6345 | + | 24% |
| Isobar-6-includes-valine-betaine- | 0.3687 | 0.6345 | − | −9% |
| 35 | | | | |
| Uridine-35 | 0.3707 | 0.6354 | − | −11% |
| Metabolite-3653-35 | 0.3724 | 0.6355 | − | −37% |
| L-anserine-35 | 0.3766 | 0.64 | − | −22% |
| Caffeine-9 | 0.3793 | 0.6403 | − | −5% |
| Metabolite-3124-35 | 0.38 | 0.6403 | + | 7% |
| pyridoxamine-phosphate-35 | 0.3852 | 0.6433 | + | 7% |
| Metabolite-2276-35 | 0.3853 | 0.6433 | − | −53% |
| Metabolite-1609-35 | 0.3866 | 0.6433 | − | −17% |
| Dulcitol-9 | 0.3897 | 0.6457 | − | −5% |
| Metabolite-344-Nonanoic acid-9 | 0.3936 | 0.6476 | + | 4% |
| Valine-9 | 0.3944 | 0.6476 | − | −14% |
| Metabolite-2366-35 | 0.3963 | 0.6476 | + | 51% |
| Metabolite-3881-35 | 0.3985 | 0.6476 | − | −12% |
| Metabolite-1514-9 | 0.3989 | 0.6476 | − | −10% |
| Glyceric acid-9 | 0.4007 | 0.6478 | − | −11% |
| Metabolite-1758-9 | 0.4068 | 0.6483 | − | −12% |
| Metabolite-3862-9 | 0.4085 | 0.6483 | − | −21% |
| Metabolite-1773-9 | 0.4098 | 0.6483 | + | 11% |
| Isobar-3-phospho-d-glycerate- | 0.4107 | 0.6483 | − | −29% |
| acetylphosphate-35 | | | | |
| Metabolite-1341-35 | 0.4107 | 0.6483 | − | −15% |
| Metabolite-2759-9 | 0.4107 | 0.6483 | − | −12% |
| Metabolite-3056-35 | 0.4218 | 0.6631 | + | 21% |
| Diaminopimelic acid-35 | 0.4263 | 0.6633 | − | −7% |
| Metabolite-3789-9 | 0.4269 | 0.6633 | − | −25% |
| Metabolite-3143-35 | 0.4376 | 0.675 | − | −20% |
| Threonine-9 | 0.441 | 0.675 | + | 16% |
| Metabolite-3734-35 | 0.4435 | 0.675 | − | −14% |
| Metabolite-2321-35 | 0.4451 | 0.675 | + | 39% |
| Metabolite-4024-35 | 0.4473 | 0.675 | − | −11% |
| Metabolite-3830-35 | 0.4482 | 0.675 | − | −48% |
| Metabolite-3652-35 | 0.449 | 0.675 | + | 50% |
| Metabolite-1775-9 | 0.453 | 0.675 | + | 16% |
| Metabolite-2193-35 | 0.4561 | 0.675 | + | 20% |
| Metabolite-2687-35 | 0.457 | 0.675 | − | −6% |
| Metabolite-3160-35 | 0.4576 | 0.675 | + | 13% |
| GABA-9 | 0.4578 | 0.675 | − | −14% |
| Alanine-9 | 0.4581 | 0.675 | + | 10% |
| Metabolite-2056-35 | 0.4633 | 0.6802 | + | 10% |
| Metabolite-4039-35 | 0.4682 | 0.684 | − | −20% |
| Metabolite-3793-9 | 0.4693 | 0.684 | + | 4% |
| Metabolite-3235-35 | 0.4718 | 0.6851 | − | −14% |
| Metabolite-2949-9 | 0.4755 | 0.688 | − | −6% |
| Metabolite-2750-35 | 0.4785 | 0.6894 | − | −27% |
| Metabolite-1573-35 | 0.4799 | 0.6894 | + | 5% |
| Metabolite-3151-9 | 0.4913 | 0.7032 | − | −10% |
| Metabolite-1125-35 | 0.4977 | 0.7069 | − | −10% |
| Inositol-9 | 0.4995 | 0.7069 | − | −16% |
| Metabolite-3594-9 | 0.5005 | 0.7069 | − | −18% |
| Metabolite-1358-9 | 0.501 | 0.7069 | − | −13% |
| Metabolite-2694-35 | 0.5031 | 0.7074 | + | 11% |
| Metabolite-3697-35 | 0.5134 | 0.7168 | + | 10% |
| Metabolite-1975-35 | 0.5246 | 0.7274 | − | −31% |
| Metabolite-2052-35 | 0.5301 | 0.7284 | − | −6% |
| gamma-L-glutamyl-L-glutamine- | 0.5309 | 0.7284 | − | −8% |
| 35 | | | | |
| Metabolite-2697-35 | 0.5322 | 0.7284 | + | 14% |
| Metabolite-293-L-Norleucine-9 | 0.5326 | 0.7284 | − | −17% |
| Metabolite-2249-35 | 0.5368 | 0.7316 | − | −11% |
| Metabolite-3994-35 | 0.5443 | 0.7373 | + | 7% |
| Glutamine-35 | 0.5466 | 0.7373 | − | −4% |
| Metabolite-1961-35 | 0.5485 | 0.7373 | − | −22% |
| Metabolite-2398-35 | 0.5485 | 0.7373 | − | −5% |
| alpha-methyl-L-beta-3-4- | 0.5516 | 0.7373 | − | −11% |
| dihydroxyphenylalanine-9 | | | | |
| Metabolite-704-9 | 0.552 | 0.7373 | − | −6% |
| Metabolite-1086-35 | 0.5549 | 0.7382 | − | −16% |
| Metabolite-1834-35 | 0.5564 | 0.7382 | − | −21% |
| Metabolite-3709-35 | 0.5603 | 0.7408 | − | −12% |
| N-5-aminocarbonyl-L-ornithine- | 0.5638 | 0.7431 | − | −10% |
| 35 | | | | |
| Metabolite-3182-35 | 0.5671 | 0.7433 | − | −10% |
| possible-D-galactose-9 | 0.5678 | 0.7433 | − | −1% |
| Isobar-2-isopropylmalic acid-4- | 0.5715 | 0.7433 | − | −7% |
| methyl-2-oxopentanoate-35 | | | | |
| Metabolite-941-9 | 0.577 | 0.7481 | + | 34% |
| possible-D-galactose-1-9 | 0.5798 | 0.7493 | + | 19% |
| Diethylstilbestrol-9 | 0.5825 | 0.7497 | − | −14% |
| Metabolite-3173-9 | 0.5838 | 0.7497 | − | −10% |
| Urea1-9 | 0.5885 | 0.7528 | − | −8% |
| alpha-aminoadipic acid-9 | 0.59 | 0.7528 | − | −8% |
| possible-D-ribose-1-9 | 0.5924 | 0.7533 | + | 16% |
| Metabolite-3951-35 | 0.5983 | 0.7581 | − | −6% |
| Metabolite-760-9 | 0.5999 | 0.7581 | − | −8% |
| Metabolite-3858-9 | 0.6026 | 0.7591 | + | 3% |
| Metabolite-1815-9 | 0.6058 | 0.7602 | + | 9% |
| Metabolite-3699-35 | 0.6082 | 0.7602 | + | 14% |
| Serine-9 | 0.6092 | 0.7602 | + | 10% |
| Metabolite-3121-9 | 0.6135 | 0.7613 | + | 12% |
| Isobar-creatine-creatinine-9 | 0.6142 | 0.7613 | + | 6% |
| Isobar-arginine-N-alpha-acetyl- | 0.6179 | 0.7613 | + | 5% |
| L-ornithine-35 | | | | |
| Metabolite-2026-35 | 0.6188 | 0.7613 | + | 10% |
| Metabolite-3740-35 | 0.6196 | 0.7613 | − | −2% |
| N-acetylserotonin-35 | 0.6277 | 0.7689 | − | −5% |
| carnosine-35 | 0.6316 | 0.7704 | − | −13% |
| Metabolite-3140-9 | 0.6363 | 0.7704 | + | 13% |
| palmitoleic acid-9 | 0.6384 | 0.7704 | − | −17% |
| Metabolite-1837-35 | 0.639 | 0.7704 | − | −5% |
| Glyoxylate-9 | 0.6395 | 0.7704 | + | 6% |
| Isobar-6-phosphogluconic acid- | 0.6426 | 0.7704 | − | −4% |
| D-arabinose-5-phosphate- | | | | |
| ribulose-5-phosphate-35 | | | | |
| Metabolite-386-9 | 0.6433 | 0.7704 | + | 15% |
| Metabolite-406-9 | 0.6448 | 0.7704 | + | 7% |
| Metabolite-2100-35 | 0.6494 | 0.7704 | + | 6% |
| Carnitine-35 | 0.6498 | 0.7704 | − | −8% |
| Histamine-35 | 0.6501 | 0.7704 | − | −6% |
| Glucarate-9 | 0.6546 | 0.7718 | + | 2% |
| Metabolite-3132-35 | 0.6552 | 0.7718 | + | 4% |
| Metabolite-2791-9 | 0.66 | 0.7752 | + | 2% |
| Metabolite-2370-35 | 0.6631 | 0.7766 | + | 12% |
| Metabolite-3805-35 | 0.6663 | 0.778 | + | 29% |
| adenosine-3-5-cyclic- | 0.6715 | 0.7818 | − | −5% |
| monophosphate-35 | | | | |
| Metabolite-3412-9 | 0.6759 | 0.7846 | + | 9% |
| possible-D-fructose-3-9 | 0.69 | 0.7987 | − | −6% |
| Metabolite-3737-35 | 0.6975 | 0.7991 | − | −6% |
| Metabolite-580-9 | 0.6992 | 0.7991 | + | 2% |
| possible-Tris-trimethylsilyl- | 0.7009 | 0.7991 | + | 2% |
| phosphate-high-concentration- | | | | |
| saturated-spectrum-9 | | | | |
| Metabolite-1753-9 | 0.7012 | 0.7991 | − | −8% |
| Metabolite-3513-35 | 0.7027 | 0.7991 | + | 8% |
| Metabolite-2151-35 | 0.7032 | 0.7991 | + | 10% |
| Metabolite-1373-9 | 0.7044 | 0.7991 | + | 6% |
| Tryptophan-35 | 0.7093 | 0.7994 | − | −3% |
| Uric acid-35 | 0.7112 | 0.7994 | − | −2% |
| Metabolite-2124-9 | 0.7139 | 0.7994 | + | 10% |
| Metabolite-404-9 | 0.7151 | 0.7994 | + | 14% |
| Metabolite-2389-35 | 0.7167 | 0.7994 | + | 8% |
| Metabolite-3579-9 | 0.7203 | 0.8012 | + | 2% |
| Metabolite-3669-35 | 0.7236 | 0.8015 | − | −2% |
| Metabolite-2111-35 | 0.7258 | 0.8015 | − | −4% |
| Metabolite-2705-9 | 0.7266 | 0.8015 | + | 14% |
| n-dodecanoate-9 | 0.7343 | 0.8048 | + | 8% |
| Metabolite-2559-35 | 0.7345 | 0.8048 | + | 24% |
| Metabolite-1335-35 | 0.7356 | 0.8048 | + | 6% |
| Metabolite-2792-9 | 0.738 | 0.8052 | + | 13% |
| L-histidinol-35 | 0.743 | 0.8077 | + | 11% |
| Leucine-9 | 0.7476 | 0.8077 | − | −7% |
| Sorbitol-35 | 0.7523 | 0.8077 | − | −3% |
| Metabolite-1353-9 | 0.7567 | 0.8077 | − | −2% |
| Metabolite-3592-9 | 0.7569 | 0.8077 | − | −7% |
| Metabolite-1776-9 | 0.757 | 0.8077 | + | 1% |
| Metabolite-3874-9 | 0.7631 | 0.8077 | − | −7% |
| Metabolite-2009-9 | 0.7639 | 0.8077 | − | −3% |
| Isobar-2-deoxy-D-galactose-2- | 0.7646 | 0.8077 | + | 8% |
| deoxy-glucose-glucarate-35 | | | | |
| Metabolite-2278-35 | 0.7663 | 0.8077 | − | −2% |
| 3-chloro-L-tyrosine-35 | 0.7725 | 0.8077 | + | 3% |
| Metabolite-3831-9 | 0.7739 | 0.8077 | + | 3% |
| Metabolite-222-9 | 0.7758 | 0.8077 | + | 1% |
| Metabolite-3659-35 | 0.776 | 0.8077 | + | 1% |
| Metabolite-3660-35 | 0.7764 | 0.8077 | − | −4% |
| Metabolite-2109-35 | 0.7775 | 0.8077 | + | 4% |
| 2-amino-heptanedioic acid-9 | 0.779 | 0.8077 | − | −5% |
| Metabolite-3135-35 | 0.7845 | 0.8077 | − | −8% |
| Metabolite-3885-9 | 0.7866 | 0.8077 | − | −3% |
| Metabolite-3666-35 | 0.7885 | 0.8077 | − | −2% |
| Metabolite-1416-9 | 0.7916 | 0.8077 | − | −22% |
| Metabolite-2027-35 | 0.7918 | 0.8077 | − | −8% |
| Metabolite-3696-35 | 0.7927 | 0.8077 | − | −5% |
| L-beta-imidazolelactic acid-35 | 0.7929 | 0.8077 | − | −5% |
| Metabolite-1534-9 | 0.796 | 0.8077 | − | −1% |
| Metabolite-1127-35 | 0.801 | 0.8077 | + | 5% |
| Metabolite-1522-9 | 0.8032 | 0.8077 | − | −1% |
| Metabolite-3451-9 | 0.8041 | 0.8077 | − | −5% |
| Isobar-adenosine-5- | 0.8043 | 0.8077 | − | −3% |
| diphosphoribose-glucosamine-6- | | | | |
| phosphate-9 | | | | |
| Metabolite-706-9 | 0.8047 | 0.8077 | + | 12% |
| Metabolite-2789-9 | 0.8048 | 0.8077 | − | −2% |
| Metabolite-3783-35 | 0.805 | 0.8077 | − | −1% |
| Aspartate-35 | 0.8084 | 0.8091 | − | −7% |
| Isoleucine-9 | 0.8111 | 0.8098 | − | −5% |
| Metabolite-2347-35 | 0.8167 | 0.8132 | + | 10% |
| Citric acid-35 | 0.8186 | 0.8132 | − | −2% |
| Octadecanoic acid-9 | 0.8312 | 0.8216 | − | −2% |
| Methionine-35 | 0.8383 | 0.8262 | − | −3% |
| Metabolite-3655-35 | 0.8418 | 0.8262 | − | −4% |
| Metabolite-1824-9 | 0.8421 | 0.8262 | + | 8% |
| Gulono-1-4-lactone-9 | 0.8474 | 0.8268 | − | −1% |
| Metabolite-3222-9 | 0.8481 | 0.8268 | + | 3% |
| Metabolite-278-9 | 0.8489 | 0.8268 | + | 5% |
| Isobar-3-methyl-L-histidine- | 0.8611 | 0.8343 | + | 6% |
| noradrenaline-35 | | | | |
| 1-7-dihydro-6h-purin-6-one-35 | 0.8624 | 0.8343 | − | −3% |
| alpha-keto-glutarate-9 | 0.8641 | 0.8343 | + | 2% |
| Proline-35 | 0.865 | 0.8343 | + | 3% |
| Metabolite-1327-35 | 0.8711 | 0.836 | − | −8% |
| Metabolite-655-9 | 0.8717 | 0.836 | + | 3% |
| DL-cystathionine-35 | 0.8738 | 0.836 | + | 1% |
| Metabolite-3575-9 | 0.8765 | 0.836 | − | −2% |
| Metabolite-2506-35 | 0.8787 | 0.836 | − | −4% |
| Metabolite-1068-35 | 0.8792 | 0.836 | − | −1% |
| Metabolite-1979-35 | 0.8956 | 0.8411 | − | −2% |
| Metabolite-734-probable- | 0.8966 | 0.8411 | + | 1% |
| aspartate-9 | | | | |
| Metabolite-1463-35 | 0.8969 | 0.8411 | − | −2% |
| Metabolite-1968-9 | 0.8988 | 0.8411 | + | 1% |
| Metabolite-2150-35 | 0.9033 | 0.8411 | − | −6% |
| Metabolite-709-9 | 0.9066 | 0.8411 | − | −3% |
| Metabolite-3224-35 | 0.9072 | 0.8411 | + | 3% |
| Metabolite-3213-9 | 0.9079 | 0.8411 | + | 2% |
| Metabolite-1754-9 | 0.9084 | 0.8411 | + | 2% |
| Metabolite-443-9 | 0.9089 | 0.8411 | − | −2% |
| Metabolite-2703-35 | 0.9116 | 0.8411 | + | 2% |
| Metabolite-3663-35 | 0.9123 | 0.8411 | + | 1% |
| Metabolite-3694-35 | 0.9131 | 0.8411 | − | −2% |
| Melatonin-9 | 0.9155 | 0.8411 | + | 2% |
| Metabolite-2221-9 | 0.9186 | 0.8411 | + | 1% |
| Metabolite-3904-9 | 0.9205 | 0.8411 | − | −2% |
| possible-sugar5-9 | 0.9211 | 0.8411 | − | −2% |
| Tyramine-9 | 0.9244 | 0.8411 | − | −2% |
| Metabolite-2628-9 | 0.9277 | 0.8411 | + | 2% |
| Metabolite-3656-35 | 0.9301 | 0.8411 | − | −1% |
| possible-sugar2-9 | 0.9306 | 0.8411 | − | −2% |
| Metabolite-1568-9 | 0.9351 | 0.8411 | + | 2% |
| Metabolite-1114-35 | 0.938 | 0.8411 | + | 1% |
| Metabolite-3910-35 | 0.9384 | 0.8411 | + | 5% |
| Metabolite-841-9 | 0.939 | 0.8411 | + | 4% |
| Metabolite-1538-9 | 0.9446 | 0.8411 | − | −1% |
| Metabolite-2788-9 | 0.9459 | 0.8411 | − | −1% |
| Metabolite-128-9 | 0.946 | 0.8411 | − | −1% |
| Metabolite-2707-9 | 0.9479 | 0.8411 | + | 1% |
| Metabolite-3977-35 | 0.9495 | 0.8411 | − | −1% |
| Metabolite-2627-9 | 0.95 | 0.8411 | + | 1% |
| Oleic acid-9 | 0.9611 | 0.8491 | + | 1% |
| Metabolite-557-9 | 0.9667 | 0.8496 | − | −2% |
| N-acetyl-L-glutamine-9 | 0.969 | 0.8496 | + | 1% |
| Metabolite-3661-35 | 0.9707 | 0.8496 | + | 0% |
| Metabolite-3701-35 | 0.972 | 0.8496 | − | 0% |
| Metabolite-499-9 | 0.9736 | 0.8496 | − | 0% |
| Metabolite-2329-35 | 0.9744 | 0.8496 | + | 1% |
| Metabolite-3704-35 | 0.9775 | 0.8504 | − | −1% |
| Metabolite-1831-35 | 0.9814 | 0.8507 | + | 1% |
| Metabolite-3732-35 | 0.9821 | 0.8507 | − | 0% |
| Metabolite-1364-35 | 0.9865 | 0.8514 | − | 0% |
| Metabolite-763-9 | 0.9872 | 0.8514 | − | 0% |
| 9,12-octadecadienoic acid-z-z-9 | 0.9944 | 0.8545 | + | 0% |
| Metabolite-1247-retired- | 0.995 | 0.8545 | + | 0% |
| unknown-3696-35 | | | | |
| Metabolite-3510-35 | 0.9994 | 0.8546 | − | 0% |

### Example 2

### Biomarkers to Distinguish AD from Controls in Females >70

In another example, biomarkers were discovered by (1) analyzing plasma samples from different groups of human subjects to determine the levels of metabolites in the samples and then (2) statistically analyzing the results to determine those metabolites that are differentially present in the two groups. The plasma samples used for the analysis were from females over 70 with Alzheimer's Disease and Control subjects (females over 70 not diagnosed with Alzheimer's Disease). After the levels of metabolites were determined, the data was analyzed using T-tests (Table 2).

As listed below in Table 2, biomarkers were discovered that were differentially present between samples from females over 70 with Alzheimer's Disease subjects and Control subjects (females over 70 not diagnosed with Alzheimer's Disease).

Table 2 includes, for each listed biomarker and non-biomarker compound, the p-value and the q-value determined in the statistical analysis of the data concerning the biomarkers, an indication of whether the mean level of a particular compound was higher in the Alzheimer's Disease or Control samples (a “+” indicating a higher mean level in Alzheimer's Disease samples as compared to the control samples and a “−” indicating a lower mean level in Alzheimer's Disease samples as compared to the control samples), and an indication of the percentage difference in the Alzheimer's Disease mean level as compared to the control mean level. Throughout the tables, names of metabolites ending with the notation “−35” indicate that the levels of those compounds were measured using LC-MS, and names ending with the notation “−9” indicate that the levels of those compounds were measured using GC-MS.

TABLE 2
AD vs Control Females over age 70

| | | | Increase | |
|---|---|---|---|---|
| | | | (+) or | % |
| | | | Decrease | Change |
| Compound | p-value | q-value | (−) in AD | in AD |
| Metabolite-3659-35 | 0 | 2.00E−04 | + | 26% |
| Metabolite-3738-35 | 2.00E−04 | 0.0368 | + | 29% |
| Metabolite-3740-35 | 5.00E−04 | 0.063 | + | 13% |
| Metabolite-3699-35 | 7.00E−04 | 0.0733 | + | 68% |
| Metabolite-3669-35 | 0.0014 | 0.1169 | + | 14% |
| Metabolite-1284-35 | 0.0018 | 0.1265 | − | −30% |
| N-carbamoyl-L-aspartate- | 0.0033 | 0.1768 | − | −38% |
| 35 | | | | |
| Metabolite-3658-35 | 0.0034 | 0.1768 | + | 10% |
| Metabolite-2506-35 | 0.0062 | 0.2853 | + | 136% |
| Urea1-9 | 0.0085 | 0.3505 | − | −21% |
| Metabolite-3218-35 | 0.0095 | 0.3543 | − | −30% |
| Metabolite-2390-35 | 0.0105 | 0.3606 | − | −37% |
| Metabolite-2185-35 | 0.0116 | 0.3691 | − | −23% |
| Metabolite-3160-35 | 0.0142 | 0.4183 | + | 27% |
| Urea2-9 | 0.0191 | 0.4336 | − | −14% |
| Metabolite-3326-35 | 0.0210 | 0.4336 | − | −25% |
| Metabolite-3816-35 | 0.0210 | 0.4336 | − | −19% |
| Metabolite-3874-9 | 0.0230 | 0.4336 | − | −22% |
| Metabolite-578-9 | 0.0230 | 0.4336 | + | 26% |
| Metabolite-3055-35 | 0.0244 | 0.4336 | − | −40% |
| Metabolite-1110-35 | 0.0253 | 0.4336 | − | −12% |
| Metabolite-3138-35 | 0.0253 | 0.4336 | − | −18% |
| Inositol-9 | 0.0302 | 0.4940 | − | −20% |
| Metabolite-2005-35 | 0.0360 | 0.4940 | − | −25% |
| Metabolite-3656-35 | 0.0360 | 0.4940 | + | 17% |
| Metabolite-1926-35 | 0.0383 | 0.4940 | − | −22% |
| Metabolite-704-9 | 0.0392 | 0.4940 | − | −15% |
| Metabolite-763-9 | 0.0392 | 0.4940 | − | −31% |
| Metabolite-841-9 | 0.0392 | 0.4940 | − | −44% |
| Metabolite-2558-35 | 0.0392 | 0.4940 | − | −34% |
| Metabolite-1069-35 | 0.0396 | 0.4940 | + | 96% |
| Metabolite-3124-35 | 0.0427 | 0.5017 | + | 10% |
| Pyridoxamine-35 | 0.0427 | 0.5017 | − | −16% |
| Metabolite-2249-35 | 0.0463 | 0.5017 | − | −26% |
| Metabolite-2269-35 | 0.0463 | 0.5017 | − | −12% |
| Metabolite-3657-35 | 0.0463 | 0.5017 | + | 8% |
| Hippuric acid-35 | 0.0503 | 0.5017 | − | −32% |
| Metabolite-1127-35 | 0.0503 | 0.5017 | + | 32% |
| Metabolite-1465-35 | 0.0503 | 0.5017 | − | −28% |
| DOPA-9 | 0.0512 | 0.5017 | − | −21% |
| Metabolite-1914-35 | 0.0587 | 0.5522 | − | −29% |

### Example 3

### Biomarkers to Distinguish AD from Controls in Males <70

In another example, biomarkers were discovered by (1) analyzing plasma samples from different groups of human subjects to determine the levels of metabolites in the samples and then (2) statistically analyzing the results to determine those metabolites that are differentially present in the two groups. The plasma samples used for the analysis were from male subjects under 70 years of age with Alzheimer's Disease and Control subjects (males under 70 years of age not diagnosed with Alzheimer's Disease). After the levels of metabolites were determined, the data was analyzed using T-tests (Table 3).

As listed below in Table 3, biomarkers were discovered that were differentially present between samples from male subjects under 70 years of age with Alzheimer's Disease subjects and Control subjects (males under 70 years of age not diagnosed with Alzheimer's Disease).

Table 3 includes, for each listed biomarker and non-biomarker compound, the p-value and the q-value determined in the statistical analysis of the data concerning the biomarkers, an indication of whether the mean level of a particular compound was higher in the Alzheimer's Disease or Control samples (a “+” indicating a higher mean level in Alzheimer's Disease samples as compared to the control samples and a “−” indicating a lower mean level in Alzheimer's Disease samples as compared to the control samples), and an indication of the percentage difference in the Alzheimer's Disease mean level as compared to the control mean level. Throughout the tables, names of metabolites ending with the notation “−35” indicate that the levels of those compounds were measured using LC-MS, and names ending with the notation “−9” indicate that the levels of those compounds were measured using GC-MS.

TABLE 3
AD vs Control Males age 70 and Under

| | | | Increase | |
|---|---|---|---|---|
| | | | (+) or | % |
| | | | Decrease | Change |
| Compound | p-value | q-value | (−) in AD | in AD |
| Metabolite-3413-9 | 2.00E−04 | 0.0577 | + | 12% |
| Caffeine-9 | 0.0022 | 0.3656 | − | −22% |
| Metabolite-565-9 | 0.0047 | 0.3656 | + | 10% |
| Metabolite-1987-9 | 0.0056 | 0.3656 | + | 12% |
| Hippuric acid-35 | 0.0056 | 0.3656 | − | −48% |
| Metabolite-1656-35 | 0.0072 | 0.3656 | − | −44% |
| Metabolite-1834-35 | 0.0072 | 0.3656 | − | −34% |
| Biliverdin-35 | 0.0079 | 0.3656 | − | −32% |
| Glutarate-9 | 0.0110 | 0.3671 | + | 43% |
| Glyoxylate-9 | 0.0111 | 0.3671 | + | 22% |
| Metabolite-1331-35 | 0.0129 | 0.3671 | − | −51% |
| Metabolite-3707-35 | 0.0129 | 0.3671 | − | −52% |
| Metabolite-3055-35 NH3 | 0.0150 | 0.3958 | − | −45% |
| adduct of Hippuric Acid | | | | |
| Metabolite-2526-35 | 0.0175 | 0.3958 | − | −28% |
| Alanine-9 | 0.0203 | 0.3958 | + | 36% |
| DL-pipecolic acid-35 | 0.0203 | 0.3958 | + | 46% |
| Metabolite-2005-35 | 0.0203 | 0.3958 | − | −24% |
| Metabolite-3765-35 | 0.0254 | 0.4083 | − | −47% |
| Metabolite-1753-9 | 0.0270 | 0.4083 | + | 54% |
| Metabolite-785-9 | 0.0270 | 0.4083 | + | 8% |
| Possible-Arabinose-3-9 | 0.0310 | 0.4083 | + | 22% |
| Metabolite-2124-9 | 0.0310 | 0.4083 | + | 58% |
| Metabolite-2791-9 | 0.0310 | 0.4083 | + | 12% |
| Metabolite-386-9 | 0.0310 | 0.4083 | + | 29% |
| Metabolite-470-9 | 0.0310 | 0.4083 | + | 15% |
| Possible-D-ribose-1-9 | 0.0327 | 0.4083 | + | 54% |
| Malic acid-9 | 0.0355 | 0.4083 | + | 33% |
| Metabolite-3633-9 | 0.0355 | 0.4083 | + | 14% |
| Metabolite-443-9 | 0.0355 | 0.4083 | + | 26% |
| Metabolite-3604-35 | 0.0378 | 0.4083 | − | −45% |
| Possible-TMS-2-t-butyl- | 0.0404 | 0.4083 | + | 31% |
| 6-methylphenol-9 | | | | |
| L-kynurenine-35 | 0.0404 | 0.4083 | − | −28% |
| Metabolite-1116-35 | 0.0404 | 0.4083 | − | −35% |
| Metabolite-3813-35 | 0.0443 | 0.4083 | − | −22% |
| Cholesterol-9 | 0.0459 | 0.4083 | + | 11% |
| Dulcitol-9 | 0.0459 | 0.4083 | + | 7% |
| Metabolite-3275-9 | 0.0459 | 0.4083 | + | 35% |
| Threonine-9 | 0.0459 | 0.4083 | + | 35% |
| Metabolite-1327-35 | 0.0459 | 0.4083 | − | −34% |
| (Bilirubin) | | | | |
| Metabolite-2722-9 | 0.0520 | 0.4083 | + | 9% |
| Metabolite-3781-35 | 0.0520 | 0.4083 | − | −17% |
| Metabolite-1974-35 | 0.0525 | 0.4083 | − | −64% |
| Metabolite-2792-9 | 0.0525 | 0.4083 | + | 165% |
| Metabolite-1866-9 | 0.0531 | 0.4083 | + | 81% |
| Metabolite-1416-9 | 0.0565 | 0.4083 | − | −54% |
| Metabolite-670-9 | 0.0588 | 0.4083 | + | 8% |
| L-anserine-35 | 0.0588 | 0.4083 | − | −41% |
| Metabolite-2269-35 | 0.0588 | 0.4083 | − | −58% |
| Metabolite-3653-35 | 0.0588 | 0.4083 | + | 212% |
| Metabolite-3762-35 | 0.0588 | 0.4083 | − | −29% |

### Example 4

### Analytical Characterization of Unnamed Biomarker Compounds

Table 4 below includes analytical characteristics of each of the unnamed metabolites listed in Tables 1, 2, and 3 above. The table includes, for each listed Metabolite, the retention time (RT), retention index (RI), mass, quant mass, and polarity obtained using the analytical methods described above. “Mass” refers to the mass of the C12 isotope of the parent ion used in quantification of the compound. The values for “Quant Mass” give an indication of the analytical method used for quantification: “Y” indicates GC-MS and “1” indicates LC-MS. “Polarity” indicates the polarity of the quantitative ion as being either positive (+) or negative (−). In some cases the Metabolite has been identified and is listed as “retired” followed with the name of the compound. In other cases the unnamed metabolite has been found to be a duplicate of another unnamed metabolite and is listed as “retired” followed with Metabolite #. In those cases future studies track that Metabolite number (e.g., Metabolite—1247—retired—Metabolite—3696, Metabolite 1247 will now be referred to as Metabolite 3696).

TABLE 4
Analytical Characteristics of Unnamed Metabolites

| | | | | Quant | |
|---|---|---|---|---|---|
| COMP_ID_NAME | RT | RI | Mass | Mass | Polarity |
| Metabolite - 1068 | 1.44 | 1490 | 203.1 | 1 | + |
| Metabolite - 1069 - retired for | 12.55 | 12930 | 367.2 | 1 | − |
| dehydroepiandrosterone sulfate | | | | | |
| Metabolite - 1086 | 4.56 | 4811 | 294.1 | 1 | + |
| Metabolite - 1110 | 11.66 | 11841 | 269.1 | 1 | − |
| Metabolite - 1111 | 2.69 | 2782 | 148.1 | 1 | + |
| Metabolite - 1113 - retired for acetylcarnitine | 4.91 | 5190 | 204.2 | 1 | + |
| Metabolite - 1114 - retired for choline | 2.19 | 2198 | 104.1 | 1 | + |
| Metabolite - 1116 - retired for (s)-2- | 4.2 | 4480 | 103.4 | 1 | − |
| hydroxybutyric acid | | | | | |
| Metabolite - 1125 | 3.94 | 4202 | 221.1 | 1 | + |
| Metabolite - 1127 | 12.18 | 12369 | 363.1 | 1 | + |
| Metabolite - 1193 - retired for 3-indoxyl | 8.85 | 9031 | 212.1 | 1 | − |
| sulfate | | | | | |
| Metabolite - 1216 | 1.6 | 1631.4 | 343.9 | 1 | − |
| Metabolite - 1247 - retired - Metabolite - 3696 | 14.8 | 14959 | 448.3 | 1 | − |
| Metabolite - 128 | 10.14 | 1697.1 | 227.171 | Y | + |
| Metabolite - 1284 | 9.71 | 9910 | 486.9 | 1 | + |
| Metabolite - 1286 | 14.41 | 14579.8 | 229 | 1 | + |
| Metabolite - 1289 | 8.96 | 9139.7 | 338.4 | 1 | + |
| Metabolite - 1323 - retired for p-cresol sulfate | 9.31 | 9719.8 | 187 | 1 | − |
| Metabolite - 1327 | 13.22 | 13705.9 | 585.4 | 1 | + |
| Metabolite - 1331 - retired for Metabolite | 12.87 | 13342.7 | 239.2 | 1 | − |
| 3707 | | | | | |
| Metabolite - 1335 | 8.74 | 9162.2 | 367.2 | 1 | + |
| Metabolite - 1341 | 2.62 | 2746.6 | 406.8 | 1 | + |
| Metabolite - 1342 | 9.04 | 9459.4 | 265.2 | 1 | + |
| Metabolite - 1345 | 13.27 | 13764.5 | 369.3 | 1 | − |
| Metabolite - 1346 | 1.27 | 1449.5 | 113 | 1 | − |
| Metabolite - 1350 | 13.75 | 14248.7 | 909.8 | 1 | + |
| Metabolite - 1353 | 13.24 | 2104.2 | 371.94 | Y | + |
| Metabolite - 1358 | 12.66 | 2038.3 | 288.015 | Y | + |
| Metabolite - 1364 | 10.35 | 10765.1 | 397.2 | 1 | + |
| Metabolite - 1368 - retired for pyridoxic acid | 8.18 | 8607.4 | 184.1 | 1 | + |
| Metabolite - 1373 | 10.26 | 1749.6 | 218.014 | Y | + |
| Metabolite - 1414 | 10.55 | 1788.9 | 259.012 | Y | + |
| Metabolite - 1416 | 11.23 | 1867.8 | 237.006 | Y | + |
| Metabolite - 1463 | 8.98 | 9186.7 | 399.1 | 1 | − |
| Metabolite - 1465 | 3.45 | 3600 | 162.1 | 1 | + |
| Metabolite - 1498 - retired duplicate of X- | 1.56 | 1650 | 143.1 | 1 | − |
| 4666 | | | | | |
| Metabolite - 1514 | 6.24 | 1239.8 | 148.068 | Y | + |
| Metabolite - 1522 | 11.43 | 1839.4 | 217.067 | Y | + |
| Metabolite - 1534 | 11.3 | 1824.3 | 246.08 | Y | + |
| Metabolite - 1538 | 10.56 | 1730.7 | 156.074 | Y | + |
| Metabolite - 1568 | 8.77 | 1542.9 | 292.055 | Y | + |
| Metabolite - 1573 - retired for glycerol 2- | 1.63 | 1669 | 170.9 | 1 | − |
| phosphate | | | | | |
| Metabolite - 1576 | 2.51 | 2530 | 247.1 | 1 | + |
| Metabolite - 1609 | 8.31 | 8529 | 378 | 1 | + |
| Metabolite - 1655 | 1.31 | 1374 | 107 | 1 | + |
| Metabolite - 1656 | 1.46 | 1509 | 154.9 | 1 | − |
| Metabolite - 1753 | 8.16 | 1446.9 | 356.938 | Y | + |
| Metabolite - 1754 | 12.64 | 1981.6 | 204.056 | Y | + |
| Metabolite - 1758 | 13.03 | 2028.7 | 203.062 | Y | + |
| Metabolite - 1773 | 11.76 | 1879.1 | 271.049 | Y | + |
| Metabolite - 1775 | 11.17 | 1809.3 | 518.181 | Y | + |
| Metabolite - 1776 | 13.11 | 2054 | 463.062 | Y | + |
| Metabolite - 1802 | 8.95 | 9328 | 486.9 | 1 | + |
| Metabolite - 1815 | 11.82 | 1886.8 | 289.079 | Y | + |
| Metabolite - 1824 | 8.55 | 1509.8 | 126.046 | Y | + |
| Metabolite - 1826 | 5.5 | 1168 | 101.993 | Y | + |
| Metabolite - 1828 | 14.5 | 2209.3 | 361.022 | Y | + |
| Metabolite - 1831 | 1.46 | 1638.7 | 209.9 | 1 | − |
| Metabolite - 1834 | 1.64 | 1794.5 | 104 | 1 | − |
| Metabolite - 1837 | 2.21 | 2315 | 216.2 | 1 | + |
| Metabolite - 1846 | 11.35 | 1833.2 | 362.028 | Y | + |
| Metabolite - 1866 | 11.67 | 1875.3 | 447.021 | Y | + |
| Metabolite - 1911 | 11.42 | 11799.6 | 464.1 | 1 | + |
| Metabolite - 1914 | 10.35 | 10719.8 | 239.1 | 1 | + |
| Metabolite - 1926 - retired - trans-2,3,4- | 11.46 | 11839.6 | 239.2 | 1 | + |
| trimethoxycinnamic acid | | | | | |
| Metabolite - 1956 probable amino malonic | 8.4 | 1482.3 | 218.037 | Y | + |
| acid - NIST | | | | | |
| Metabolite - 1957 | 10.83 | 1771.5 | 218.042 | Y | + |
| Metabolite - 1961 - retired - glycocholic acid | 14.02 | 14430.7 | 466.1 | 1 | + |
| Metabolite - 1968 | 10.86 | 1778.2 | 414.045 | Y | + |
| Metabolite - 1974 | 5.93 | 6077 | 160.2 | 1 | + |
| Metabolite - 1975 | 5.95 | 6093 | 344 | 1 | + |
| Metabolite - 1979 - retired Cl adduct of isobar | 1.52 | 1690.3 | 199 | 1 | − |
| 19 | | | | | |
| Metabolite - 1981 | 7.94 | 8266.8 | 158.1 | 1 | + |
| Metabolite - 1987 | 13.22 | 2056.6 | 217.026 | Y | + |
| Metabolite - 1988 - retired for 3- | 11.14 | 11515 | 190.1 | 1 | + |
| indolepropionate | | | | | |
| Metabolite - 2005 | 8.62 | 9048 | 232.1 | 1 | + |
| Metabolite - 2008 | 16.28 | 16711.4 | 254.4 | 1 | + |
| Metabolite - 2009 | 11.93 | 1905.9 | 217 | Y | + |
| Metabolite - 2026 - retired for Isobar 55 | 1.36 | 1556.2 | 239.2 | 1 | + |
| Metabolite - 2027 | 1.56 | 1729.3 | 184.1 | 1 | + |
| Metabolite - 2041 | 13.84 | 14198.1 | 246.3 | 1 | + |
| Metabolite - 2052 - retired - K adduct of | 1.3 | 1429.8 | 219.1 | 1 | + |
| isobar 01 | | | | | |
| Metabolite - 2056 - retired for erythrose | 1.37 | 1499 | 165.1 | 1 | − |
| Metabolite - 2074 | 2.24 | 2380.9 | 280.1 | 1 | + |
| Metabolite - 2100 - retired Na adduct of | 1.33 | 1532.9 | 499 | 1 | + |
| Isobar 55 dimer | | | | | |
| Metabolite - 2109 | 8.99 | 9266 | 321.1 | 1 | + |
| Metabolite - 2111 | 9.19 | 9442.3 | 365.1 | 1 | + |
| Metabolite - 2113 | 8.32 | 1476.1 | 332.073 | Y | + |
| Metabolite - 2124 | 10.49 | 1731.3 | 274.003 | Y | + |
| Metabolite - 2139 - retired for | 8.09 | 8416.7 | 218.1 | 1 | + |
| propionylcarnitine | | | | | |
| Metabolite - 2150 | 13.27 | 13616.5 | 466.1 | 1 | + |
| Metabolite - 2151 | 14.43 | 14721.8 | 531.3 | 1 | + |
| Metabolite - 2173 | 2.68 | 2748.2 | 230.1 | 1 | + |
| Metabolite - 2185 - isovaleryl-, valeryl- and/or | 9.22 | 9499.4 | 246.2 | 1 | + |
| 2-methylbutytl- carnitine | | | | | |
| Metabolite - 2193 | 8.39 | 8699 | 233.1 | 1 | + |
| Metabolite - 222 | 11.02 | 1835.7 | 319.092 | Y | + |
| Metabolite - 2220 TMS-pyrophosphate | 9.75 | 1639.3 | 450.927 | Y | + |
| Metabolite - 2221 | 11.57 | 1862.5 | 308.112 | Y | + |
| Metabolite - 2249 | 14.21 | 14570.9 | 267.2 | 1 | − |
| Metabolite - 2250 | 14.26 | 14668.4 | 286.3 | 1 | + |
| Metabolite - 2258 | 11.09 | 11425 | 286.3 | 1 | + |
| Metabolite - 2269 | 10.36 | 10727 | 255.1 | 1 | − |
| Metabolite - 2270 | 11.03 | 11401.8 | 495.2 | 1 | − |
| Metabolite - 2273 | 9.28 | 9643.2 | 586.5 | 1 | + |
| Metabolite - 2276 | 9.78 | 10129.3 | 199 | 1 | − |
| Metabolite - 2278 | 10.34 | 10690.7 | 308.1 | 1 | + |
| Metabolite - 2285 | 2 | 2146 | 699.6 | 1 | − |
| Metabolite - 2291 | 10.55 | 10921 | 213.1 | 1 | − |
| Metabolite - 2306 - retired - gamma-glu-leu | 8.89 | 9246 | 261.1 | 1 | + |
| Metabolite - 2313 | 1.56 | 1685.6 | 352.9 | 1 | − |
| Metabolite - 2320 | 12.27 | 12640 | 288.3 | 1 | + |
| Metabolite - 2321 | 13.44 | 13832.6 | 314.3 | 1 | + |
| Metabolite - 2322 | 14.61 | 15056.5 | 616.3 | 1 | + |
| Metabolite - 2329 | 11.76 | 12177.6 | 541.2 | 1 | − |
| Metabolite - 2347 | 13.65 | 14091 | 450.1 | 1 | + |
| Metabolite - 2348 | 13.91 | 14293.5 | 448.3 | 1 | + |
| Metabolite - 2366 | 8.47 | 8870.2 | 271 | 1 | + |
| Metabolite - 2370 | 16.13 | 16561.2 | 476.4 | 1 | − |
| Metabolite - 2386 | 11.94 | 12320.3 | 539.2 | 1 | − |
| Metabolite - 2389 | 1.49 | 1641.5 | 314.9 | 1 | − |
| Metabolite - 2390 | 6.09 | 6144.9 | 517.4 | 1 | + |
| Metabolite - 2398 | 13.07 | 13405.8 | 404 | 1 | + |
| Metabolite - 2426 | 8.92 | 9236 | 129.2 | 1 | − |
| Metabolite - 2506 | 14.05 | 14437.5 | 624.4 | 1 | − |
| Metabolite - 2526 | 1.38 | 1516 | 215 | 1 | − |
| Metabolite - 2548 - retired Cl adduct of uric | 5.97 | 6016 | 202.9 | 1 | − |
| acid | | | | | |
| Metabolite - 2558 | 8.14 | 8674 | 153.1 | 1 | + |
| Metabolite - 2559 | 13.83 | 14151 | 539.2 | 1 | − |
| Metabolite - 2561 | 10.2 | 10481 | 352.1 | 1 | + |
| Metabolite - 2567 - retired for glutamyl-valine | 7.79 | 8164.7 | 247.1 | 1 | + |
| Metabolite - 261 | 17.2 | 2506.8 | 311.325 | Y | + |
| Metabolite - 2627 | 9.19 | 1601.6 | 334.058 | Y | + |
| Metabolite - 2628 | 12.51 | 2000.2 | 308.035 | Y | + |
| Metabolite - 268 | 8.1 | 1507.6 | 144.159 | Y | + |
| Metabolite - 2687 - retired Cl adduct of | 1.4 | 1593 | 181.1 | 1 | − |
| glutamine | | | | | |
| Metabolite - 2693 | 1.95 | 2067.3 | 258.1 | 1 | + |
| Metabolite - 2694 - retired lactic acid | 2.23 | 2321 | 135 | 1 | − |
| Metabolite - 2697 | 3.77 | 4241.2 | 209.9 | 1 | + |
| Metabolite - 2703 | 8.86 | 9054.8 | 384.1 | 1 | + |
| Metabolite - 2705 | 8.76 | 1587.3 | 344.036 | Y | + |
| Metabolite - 2707 | 8.81 | 1594.5 | 210.911 | Y | + |
| Metabolite - 2722 | 14.92 | 2329 | 137.071 | Y | + |
| Metabolite - 2750 - retired for 3316 | 2.17 | 2260 | 125.6 | 1 | − |
| Metabolite - 2753 | 3.38 | 3358 | 147 | 1 | + |
| Metabolite - 2759 | 8.43 | 1546.3 | 246.048 | Y | + |
| Metabolite - 2760 | 9.36 | 1654.4 | 320.094 | Y | + |
| Metabolite - 2774 | 3.53 | 3796 | 230.9 | 1 | + |
| Metabolite - 278 | 9.1 | 1624.2 | 117.074 | Y | + |
| Metabolite - 2788 | 10.92 | 1783.7 | 335.039 | Y | + |
| Metabolite - 2789 | 12.24 | 1953.3 | 243 | Y | + |
| Metabolite - 2790 | 13.04 | 2049.8 | 246.024 | Y | + |
| Metabolite - 2791 | 13.31 | 2067.8 | 217 | Y | + |
| Metabolite - 2792 | 13.49 | 2104.1 | 406.029 | Y | + |
| Metabolite - 2806 | 1.38 | 1491 | 185.1 | 1 | + |
| Metabolite - 2821 | 6.8 | 6913 | 119.1 | 1 | + |
| Metabolite - 287 | 12.68 | 2045 | 299.107 | Y | + |
| Metabolite - 2894 | 9.94 | 10320 | 226.1 | 1 | − |
| Metabolite - 2895 | 10.33 | 10620 | 284.1 | 1 | + |
| Metabolite - 2949 | 10.69 | 1773.7 | 319.109 | Y | + |
| Metabolite - 3044 | 1.52 | 1615.3 | 150.1 | 1 | + |
| Metabolite - 3052 | 8.7 | 8913.4 | 426.2 | 1 | + |
| Metabolite - 3055 - retired - NH3 adduct of | 9.2 | 9443 | 196.8 | 1 | + |
| hippuric acid | | | | | |
| Metabolite - 3056 | 9.19 | 9432 | 185.2 | 1 | + |
| Metabolite - 3072 | 16.23 | 16343.4 | 282.3 | 1 | + |
| Metabolite - 3121 | 4.57 | 1080.1 | 110.037 | Y | + |
| Metabolite - 3124 | 4.17 | 4545.7 | 307.1 | 1 | + |
| Metabolite - 3130 | 9.09 | 9328 | 158.2 | 1 | + |
| Metabolite - 3131 - retired NH4 adduct of | 10.49 | 10770 | 192.9 | 1 | + |
| indole-3-acetic acid | | | | | |
| Metabolite - 3132 - retired for DL-hexanoyl- | 10.14 | 10392 | 260.2 | 1 | + |
| carnitine | | | | | |
| Metabolite - 3134 - retired NH4 adduct of | 14.33 | 14487.3 | 483.1 | 1 | + |
| glycocholate | | | | | |
| Metabolite - 3135 - retired NH4 adduct of | 14.96 | 15107.7 | 467.2 | 1 | + |
| Isobar 66 | | | | | |
| Metabolite - 3138 - retired for pro-leu | 8.63 | 8749 | 229.2 | 1 | + |
| Metabolite - 3139 | 8.82 | 8934.5 | 176.1 | 1 | + |
| Metabolite - 3140 | 9.29 | 1609.2 | 262.053 | Y | + |
| Metabolite - 3143 | 9.81 | 10070 | 160.1 | 1 | + |
| Metabolite - 3151 | 6.21 | 1258.9 | 210.887 | Y | + |
| Metabolite - 3160 | 12.11 | 12247.3 | 361 | 1 | + |
| Metabolite - 3165 | 8.38 | 8472.2 | 265 | 1 | + |
| Metabolite - 3170 | 11.45 | 1871.6 | 333.053 | Y | + |
| Metabolite - 3173 | 11.76 | 1906.6 | 742.83 | Y | + |
| Metabolite - 3178 - retired NH3 adduct of | 3.15 | 3280 | 210 | 1 | + |
| isobar 42 | | | | | |
| Metabolite - 3181 | 8.59 | 8621.4 | 165.1 | 1 | + |
| Metabolite - 3182 | 8.83 | 8971 | 332.7 | 1 | + |
| Metabolite - 3183 - retired for gamma- | 9.37 | 9441 | 295.2 | 1 | + |
| glutamylphenylalanine | | | | | |
| Metabolite - 3211 | 4.77 | 1108.6 | 131.025 | Y | + |
| Metabolite - 3213 | 9.27 | 1609.6 | 188.016 | Y | + |
| Metabolite - 3218 | 2.2 | 2257 | 148.1 | 1 | + |
| Metabolite - 3222 | 11.49 | 1876.6 | 203.003 | Y | + |
| Metabolite - 3224 | 9.54 | 9634.3 | 392.8 | 1 | + |
| Metabolite - 3230 | 3.1 | 3043.2 | 245 | 1 | + |
| Metabolite - 3235 - retired - DL-indole-3- | 10.54 | 10581.1 | 206 | 1 | + |
| lactic acid | | | | | |
| Metabolite - 3252 | 6.39 | 1287.3 | 178.972 | Y | + |
| Metabolite - 3275 | 9.32 | 1626.8 | 520.908 | Y | + |
| Metabolite - 3282 | 10.07 | 1714.6 | 356.894 | Y | + |
| Metabolite - 3312 | 13.67 | 13911 | 241.2 | 1 | + |
| Metabolite - 3313 | 8.1 | 8529.6 | 196.9 | 1 | − |
| Metabolite - 3326 | 9.92 | 10173.7 | 487 | 1 | + |
| Metabolite - 3327 | 11.56 | 11784 | 385.3 | 1 | − |
| Metabolite - 3334 | 3.15 | 3371.5 | 409 | 1 | + |
| Metabolite - 3370 | 8.11 | 8529.1 | 226.2 | 1 | + |
| Metabolite - 3412 | 12.65 | 2021.5 | 216.995 | Y | + |
| Metabolite - 3413 | 12.77 | 2035.4 | 174.031 | Y | + |
| Metabolite - 3451 | 11.9 | 1931.5 | 289.989 | Y | + |
| Metabolite - 3469 - retired dimer of isobar 6 | 2.17 | 2240.8 | 235.2 | 1 | + |
| Metabolite - 3474 | 15.67 | 16524.3 | 228.3 | 1 | + |
| Metabolite - 3475 | 1.66 | 1711.9 | 365.2 | 1 | + |
| Metabolite - 3510 | 10.12 | 10739.3 | 389.4 | 1 | + |
| Metabolite - 3513 | 10.23 | 10849 | 369.3 | 1 | + |
| Metabolite - 3575 | 10.4 | 1763 | 348 | Y | + |
| Metabolite - 3579 | 10.68 | 1797 | 169 | Y | + |
| Metabolite - 3592 | 12.57 | 2016 | 204 | Y | + |
| Metabolite - 3594 | 12.72 | 2034.1 | 449.041 | Y | + |
| Metabolite - 3604 - retired Cl adduct of | 8.99 | 9551.9 | 214.2 | 1 | − |
| hippuric acid | | | | | |
| Metabolite - 3633 | 14.88 | 2304 | 133.003 | Y | + |
| Metabolite - 3651 | 1.36 | 1477.8 | 205.1 | 1 | + |
| Metabolite - 3652 | 2.05 | 2172.2 | 202 | 1 | + |
| Metabolite - 3653 | 4.05 | 4500 | 144.1 | 1 | + |
| Metabolite - 3655 | 8.72 | 8770.4 | 299.1 | 1 | + |
| Metabolite - 3656 | 9.26 | 9466 | 204.1 | 1 | + |
| Metabolite - 3657 | 9.99 | 10262.5 | 429.3 | 1 | + |
| Metabolite - 3658 | 10.22 | 10419 | 459.2 | 1 | + |
| Metabolite - 3659 | 10.28 | 10447.6 | 427.2 | 1 | + |
| Metabolite - 3660 | 10.32 | 10622.4 | 387.1 | 1 | + |
| Metabolite - 3661 | 10.49 | 10825 | 427.2 | 1 | + |
| Metabolite - 3663 - retired for isoxanthopterin | 8.4 | 8649 | 180.1 | 1 | + |
| Metabolite - 3664 | 8.72 | 8784.7 | 264.8 | 1 | + |
| Metabolite - 3665 | 8.74 | 8946 | 385.1 | 1 | + |
| Metabolite - 3666 | 9.12 | 9210 | 230 | 1 | + |
| Metabolite - 3667 | 9.17 | 9410.6 | 301.1 | 1 | + |
| Metabolite - 3669 | 9.91 | 10100 | 427 | 1 | + |
| Metabolite - 3692 | 4.2 | 4822.6 | 186 | 1 | + |
| Metabolite - 3694 | 8.05 | 8483.7 | 364.1 | 1 | + |
| Metabolite - 3696 - retired - isobar | 14.96 | 15200 | 450.3 | 1 | + |
| glycochenodeoxycholic | | | | | |
| acid/glycodeoxycholic acid | | | | | |
| Metabolite - 3697 | 7.79 | 8336.5 | 259 | 1 | + |
| Metabolite - 3698 | 8.31 | 8640.2 | 273.1 | 1 | + |
| Metabolite - 3699 | 9.97 | 10259.5 | 445.2 | 1 | + |
| Metabolite - 3701 | 1.34 | 1455.6 | 141.2 | 1 | + |
| Metabolite - 3704 | 9.01 | 9176.3 | 183.2 | 1 | + |
| Metabolite - 3705 | 9.86 | 10159.5 | 366.2 | 1 | + |
| Metabolite - 3707 retired for 3-carboxy-4- | 13.07 | 13339.5 | 241 | 1 | + |
| Methyl-5-propyl-2 furanpropanoic acid | | | | | |
| Metabolite - 3708 | 1.66 | 1625.3 | 159.9 | 1 | + |
| Metabolite - 3709 | 1.74 | 1828.2 | 202 | 1 | + |
| Metabolite - 3710 | 2.12 | 2174.3 | 491.8 | 1 | − |
| Metabolite - 3732 | 4.28 | 4921.5 | 184.1 | 1 | + |
| Metabolite - 3733 | 2.69 | 2775.3 | 317.1 | 1 | + |
| Metabolite - 3734 | 9.84 | 9973.6 | 315.1 | 1 | + |
| Metabolite - 3737 - retired - acetylcholine | 4.66 | 5221 | 146.1 | 1 | + |
| Metabolite - 3738 | 7.97 | 8283 | 366.1 | 1 | + |
| Metabolite - 3739 | 15.79 | 16200.3 | 256.4 | 1 | + |
| Metabolite - 3740 | 10.23 | 10458 | 503 | 1 | − |
| Metabolite - 3752 | 8.61 | 8750.4 | 276.1 | 1 | + |
| Metabolite - 3754 | 9.02 | 9152.5 | 190.2 | 1 | + |
| Metabolite - 3760 | 3.78 | 4445 | 162.1 | 1 | + |
| Metabolite - 3762 | 10.24 | 10504.5 | 485.3 | 1 | + |
| Metabolite - 3765 | 9.22 | 9420 | 467.8 | 1 | + |
| Metabolite - 3766 | 10.36 | 10643.8 | 398.9 | 1 | + |
| Metabolite - 3781 - retired Na adduct of | 1.45 | 1544 | 262.9 | 1 | + |
| Isobar 21 | | | | | |
| Metabolite - 3783 | 1.37 | 1464 | 271.1 | 1 | + |
| Metabolite - 3789 | 11.2 | 1843 | 279.985 | Y | + |
| Metabolite - 3793 | 10.74 | 1793.7 | 434.905 | Y | + |
| Metabolite - 3794 | 11.06 | 1832.8 | 319.067 | Y | + |
| Metabolite - 3797 | 12.6 | 2010.1 | 596.92 | Y | + |
| Metabolite - 3805 | 2.49 | 2794 | 229.1 | 1 | + |
| Metabolite - 3810 | 3.74 | 4241.5 | 188.1 | 1 | − |
| Metabolite - 3813 | 3.81 | 4312 | 212.1 | 1 | + |
| Metabolite - 3816 - retired for trans/cis- | 4.16 | 4350 | 173.1 | 1 | − |
| aconitic acid-1 | | | | | |
| Metabolite - 3830 | 8.42 | 8725 | 189 | 1 | − |
| Metabolite - 3831 | 8.6 | 1535 | 314 | Y | + |
| Metabolite - 3843 | 9.54 | 9721.9 | 263.1 | 1 | + |
| Metabolite - 3858 | 10.74 | 1791 | 206 | Y | + |
| Metabolite - 386 | 9.3 | 1580.1 | 142.092 | Y | + |
| Metabolite - 3862 | 11.4 | 1869.9 | 174.04 | Y | + |
| Metabolite - 3874 | 12.06 | 1946.7 | 457.99 | Y | + |
| Metabolite - 3881 - retired for azelaic acid | 11.05 | 11318.4 | 188.9 | 1 | + |
| Metabolite - 3885 | 12.49 | 1998.7 | 405.95 | Y | + |
| Metabolite - 3904 | 12.6 | 2011.3 | 204.002 | Y | + |
| Metabolite - 3905 | 12.76 | 2029.1 | 374.935 | Y | + |
| Metabolite - 3910 | 8.27 | 8562.2 | 267.2 | 1 | + |
| Metabolite - 3951 | 8.41 | 8705.4 | 367.1 | 1 | + |
| Metabolite - 3956 - retired for p-acetamido- | 9.15 | 9349.4 | 328 | 1 | + |
| beta-D-glucuronide | | | | | |
| Metabolite - 3977 | 11.03 | 11312 | 187.1 | 1 | − |
| Metabolite - 3992 | 1.4 | 1400 | 127.2 | 1 | − |
| Metabolite - 3994 | 1.63 | 1640.4 | 427 | 1 | + |
| Metabolite - 4003 | 3.94 | 4397 | 205 | 1 | + |
| Metabolite - 4024 | 8.58 | 8743.3 | 524.8 | 1 | − |
| Metabolite - 4039 | 15.55 | 15829.3 | 298.3 | 1 | + |
| Metabolite - 404 | 9.34 | 1584.2 | 227.098 | Y | + |
| Metabolite - 406 | 11.8 | 1883.2 | 204.074 | Y | + |
| Metabolite - 407 | 16.65 | 2483.2 | 283.2 | Y | + |
| Metabolite - 4114 | 8.73 | 8913.4 | 549.2 | 1 | + |
| Metabolite - 443 | 8.66 | 1506.6 | 205.092 | Y | + |
| Metabolite - 451 | 5.21 | 1118.4 | 173.075 | Y | + |
| Metabolite - 470 | 15.4 | 2329.7 | 299.05 | Y | + |
| Metabolite - 485 | 5.31 | 1128.8 | 217.029 | Y | + |
| Metabolite - 499 | 6.64 | 1276.1 | 259.062 | Y | + |
| Metabolite - 543 | 12.72 | 2006.8 | 117.082 | Y | + |
| Metabolite - 557 | 10.85 | 1784.5 | 245.094 | Y | + |
| Metabolite - 565 | 13.33 | 2078.4 | 129.09 | Y | + |
| Metabolite - 578 | 12.49 | 1979.2 | 498.188 | Y | + |
| Metabolite - 580 | 11.62 | 1876.8 | 102.025 | Y | + |
| Metabolite - 594 | 11.51 | 1871.3 | 205.116 | Y | + |
| Metabolite - 614 | 5.8 | 1196.7 | 233.092 | Y | + |
| Metabolite - 655 | 11.74 | 1912.8 | 156.085 | Y | + |
| Metabolite - 669 | 12.69 | 2017.7 | 361.164 | Y | + |
| Metabolite - 670 | 12.88 | 2039.7 | 258.125 | Y | + |
| Metabolite - 704 | 6.51 | 1275.1 | 171.059 | Y | + |
| Metabolite - 706 | 10.55 | 1751.4 | 437.153 | Y | + |
| Metabolite - 709 | 12.5 | 1979.8 | 109.065 | Y | + |
| Metabolite - 734 - probable aspartate | 8.42 | 1524.1 | 232.108 | Y | + |
| Metabolite - 760 | 8.67 | 1552.4 | 220.099 | Y | + |
| Metabolite - 763 | 8.45 | 1528.5 | 230.135 | Y | + |
| Metabolite - 785 | 13.33 | 2080.5 | 357.209 | Y | + |
| Metabolite - 841 | 15.01 | 2261.1 | 488.377 | Y | + |
| Metabolite - 941 | 11.6 | 1912.5 | 204.1 | Y | + |

While the invention has been described in detail and with reference to specific embodiments thereof, it will be apparent to one skilled in the art that various changes and modifications can be made without departing from the spirit and scope of the invention.

What is claimed is:

1. A method of diagnosing whether a subject has Alzheimer's Disease (AD), comprising:
analyzing a biological sample from a subject to determine the level(s) of one or more biomarkers for Alzheimer's Disease in the sample, wherein the one or more biomarkers are selected from Tables 1, 2 and/or 3; and
comparing the level(s) of the one or more biomarkers in the sample to Alzheimer's Disease-positive and/or Alzheimer's Disease-negative reference levels of the one or more biomarkers in order to diagnose whether the subject has Alzheimer's Disease.

2. The method of claim 1, wherein the one or more biomarkers are selected from those biomarkers in Tables 1, 2, and/or 3 having p values of less than 0.05 and/or those biomarkers in Tables 1, 2, and/or 3 having q values of less than 0.10.

3. The method of claim 1, wherein the Alzheimer's Disease-negative reference levels of the one or more biomarkers comprise levels of the one or more biomarkers in one or more samples from one or more subjects not having Alzheimer's Disease and the Alzheimer's Disease-positive reference levels of the one or more biomarkers comprise levels of the one or more biomarkers in one or more samples from one or more subjects diagnosed with Alzheimer's Disease.

4. The method of claim 3, wherein differential levels of the one or more biomarkers between the sample and the Alzheimer's Disease-negative reference levels are indicative of a diagnosis of Alzheimer's Disease in the subject.

5. The method of claim 3, wherein differential levels of the one or more biomarkers between the sample and the Alzheimer's Disease-positive reference levels are indicative of a diagnosis of no Alzheimer's Disease in the subject.

6. The method of claim 3, wherein levels of the one or more biomarkers in the sample corresponding to the Alzheimer's Disease-positive reference levels are indicative of a diagnosis of Alzheimer's Disease in the subject.

6. The method of claim 3, wherein levels of the one or more biomarkers in the sample corresponding to the Alzheimer's Disease-negative reference levels are indicative of a diagnosis of no Alzheimer's Disease in the subject.

7. The method of claim 1, wherein the method comprises analyzing the biological sample to determine the level of two or more biomarkers selected from Tables 1, 2, and/or 3.

8. The method of claim 1, wherein the method comprises analyzing the biological sample to determine the level of five or more biomarkers selected from Tables 1, 2, and/or 3.

9. The method of claim 1, wherein the method comprises analyzing the biological sample to determine the level of ten or more biomarkers selected from Tables 1, 2, and/or 3.

10. The method of claim 1, wherein the biological sample is cerebral spinal fluid and the one or more biomarkers are selected from Tables 1, 2, and/or 3.

11. The method of claim 1, wherein the biological sample is blood plasma and the one or more biomarkers are selected from Tables 1, 2, and/or 3.

12. The method of claim 1, wherein the sample is analyzed using one or more techniques selected from the group consisting of mass spectrometry, ELISA, and antibody linkage.

13. A method of determining whether a subject is predisposed to developing Alzheimer's Disease (AD), comprising:
analyzing a biological sample from a subject to determine the level(s) of one or more biomarkers for Alzheimer's Disease in the sample, wherein the one or more biomarkers are selected from Tables 1, 2, and/or 3; and
comparing the level(s) of the one or more biomarkers in the sample to Alzheimer's Disease-positive and/or Alzheimer's Disease-negative reference levels of the one or more biomarkers in order to determine whether the subject is predisposed to developing Alzheimer's Disease.

14. A method of monitoring progression/regression of Alzheimer's Disease (AD) in a subject comprising:
analyzing a first biological sample from a subject to determine the level(s) of one or more biomarkers for Alzheimer's Disease in the sample, wherein the one or more biomarkers are selected from Tables 1, 2, and/or 3 and the first sample is obtained from the subject at a first time point;
analyzing a second biological sample from a subject to determine the level(s) of the one or more biomarkers, wherein the second sample is obtained from the subject at a second time point; and
comparing the level(s) of one or more biomarkers in the first sample to the level(s) of the one or more biomarkers in the second sample in order to monitor the progression/regression of Alzheimer's Disease in the subject.

15. The method of claim 14, wherein the method further comprises comparing the level(s) of one or more biomarkers in the first sample, the level(s) of one or more biomarkers in the second sample, and/or the results of the comparison of the level(s) of the one or more biomarkers in the first and second samples to Alzheimer's Disease-positive and/or Alzheimer's Disease-negative reference levels of the one or more biomarkers.

16. A method of assessing the efficacy of a composition for treating Alzheimer's Disease (AD) comprising:
analyzing, from a subject having Alzheimer's Disease and currently or previously being treated with a composition, a biological sample to determine the level(s) of one or more biomarkers for Alzheimer's Disease selected from Tables 1, 2, and/or 3; and
comparing the level(s) of the one or more biomarkers in the sample to (a) levels of the one or more biomarkers in a previously-taken biological sample from the subject, wherein the previously-taken biological sample was obtained from the subject before being treated with the composition, (b) Alzheimer's Disease-positive reference levels of the one or more biomarkers, and/or (c) Alzheimer's Disease-negative reference levels of the one or more biomarkers.

17. A method for assessing the efficacy of a composition in treating Alzheimer's Disease (AD), comprising:
analyzing a first biological sample from a subject to determine the level(s) of one or more biomarkers for Alzheimer's Disease selected from Tables 1, 2, and/or 3, the first sample obtained from the subject at a first time point;
administering the composition to the subject;
analyzing a second biological sample from the subject to determine the level(s) of the one or more biomarkers, the second sample obtained from the subject at a second time point after administration of the composition;
comparing the level(s) of one or more biomarkers in the first sample to the level(s) of the one or more biomarkers in the second sample in order to assess the efficacy of the composition for treating Alzheimer's Disease.

18. A method of assessing the relative efficacy of two or more compositions for treating Alzheimer's Disease (AD) comprising:
analyzing, from a first subject having Alzheimer's Disease and currently or previously being treated with a first composition, a first biological sample to determine the level(s) of one or more biomarkers selected from Tables 1, 2, and/or 3;
analyzing, from a second subject having Alzheimer's Disease and currently or previously being treated with a second composition, a second biological sample to determine the level(s) of the one or more biomarkers; and
comparing the level(s) of one or more biomarkers in the first sample to the level(s) of the one or more biomarkers in the second sample in order to assess the relative efficacy of the first and second compositions for treating Alzheimer's Disease.

19. A method for screening a composition for activity in modulating one or more biomarkers of Alzheimer's Disease, comprising:
contacting one or more cells with a composition;
analyzing at least a portion of the one or more cells or a biological sample associated with the cells to determine the level(s) of one or more biomarkers of Alzheimer's Disease selected from Tables 1, 2, and/or 3; and
comparing the level(s) of the one or more biomarkers with predetermined standard levels for the biomarkers to determine whether the composition modulated the level(s) of the one or more biomarkers.

20. The method of claim 19 wherein the predetermined standard levels for the biomarkers are level(s) of the one or more biomarkers in the one or more cells in the absence of the composition.

21. The method of claim 19, wherein the predetermined standard levels for the biomarkers are level(s) of the one or more biomarkers in one or more control cells not contacted with the composition.

22. The method of claim 19, wherein the method is conducted in vivo.

23. The method of claim 19, wherein the method is conducted in vitro.

24. A method for identifying a potential drug target for Alzheimer's Disease (AD) comprising:
identifying one or more biochemical pathways associated with one or more biomarkers for Alzheimer's Disease selected from Tables 1, 2, and/or 3; and
identifying a protein affecting at least one of the one or more identified biochemical pathways, the protein being a potential drug target for Alzheimer's Disease.

25. A method for treating a subject having Alzheimer's Disease (AD) comprising administering to the subject an effective amount of one or more biomarkers selected from Tables 1, 2, and/or 3 that are decreased in Alzheimer's Disease.
